(19) United States
(12) Patent Application Publication
(10) Pub. No.: US 2024/0277597 A1
(43) Pub. Date: Aug. 22, 2024

(54) SUSPENDING POLYMER COMPOSITION AND METHOD OF USE THEREOF

(71) Applicant: ISP Investments LLC, Wilmington, DE (US)

(72) Inventors: Gijsbert KROON, Giessenburg (NL); Tuttu Maria NUUTINEN, Delft (NL); Eric-Jan de FEIJ, Alphen aan den Rij (NL)

(73) Assignee: ISP Investments LLC, Wilmington, DE (US)

(21) Appl. No.: 18/571,558

(22) PCT Filed: Jun. 15, 2022
(86) PCT No.: PCT/US22/33522

§ 371 Date: Dec. 18, 2023

Related U.S. Application Data
(60) Provisional application No. 63/211,201, filed on Jun. 16, 2021.

(51) Int. Cl. A61K 8/73; A61Q 5/02

(52) U.S. Cl. CPC A61K 8/731; A61Q 5/02; A61K 2800/30; A61K 2800/5922

(57) ABSTRACT

The present application relates to a suspending polymer composition comprising a blend of: (i) about 50 wt. % to about 75 wt. % of hydroxy propylmethylcellulose (HPMC), (ii) about 10 wt. % to about 25 wt. % of xanthan gum, and (iii) about 10 wt. % to about 25 wt. % of carboxymethyl cellulose (CMC). The composition is particularly useful in personal care and home care compositions. The carboxymethyl cellulose (CMC) has a degree of substitution (DS) value of less than 0.65.

### FIELD OF THE INVENTION

The present application relates to a polymer composition, and more particularly, to a suspending polymer composition comprising a blend of: (i) hydroxypropylmethylcellulose (HPMC); (ii) xanthan gum; and (iii) carboxymethyl cellulose (CMC). The suspending polymer composition finds particular utility in personal care and home care compositions.

### BACKGROUND OF THE INVENTION

Various substantially insoluble compounds cannot be adequately stabilized in an aqueous surfactant containing composition. For example, specific aqueous surfactant formulations that have silicone additives in them such as personal care and/or home care compositions isolate from their surfactant base. Conventional polymeric rheology modifiers such as carbomers or acrylic acid polymers, alkyl acrylate cross polymers, etc., have been widely used to improve viscosity of such formulations.

There are naturally derived technologies also available in the market such as Xanthan Gum, Bentonite, Hectorite, Cellulose gum and Guar. The challenge is that most of the technologies provide only thickening but not suspension performance. The ingredients which suspend can have a negative influence on sensorial benefits (rheology, foam) and silicone deposition. Also, the stability of the composition can be an issue. Currently no single ingredient offers the desired suspension stability and other related characteristics discussed above.

US Pat. Application 2004/0,158,058 assigned to Hercules LLC., discloses a composition comprising carboxymethylcellulose (CMC) having relative urea/water ratio of less than 0.9 that exhibits associative and thixotropic properties. This document further discloses a blended composition of CMC and another water soluble or water swellable polymer selected from the group consisting of polysaccharides, biopolymers, synthetic polymers, and thickening silicas.

US Pat. Application 2008/0,071,077 assigned to Akzo Nobel NV, describes a cosmetic formulation containing carboxymethyl cellulose which forms a gel at 25° C. after high shear dissolution in 0.3 wt. % aqueous sodium chloride solution. This document further discloses the use of such CMC and one or more conventional thickening agents such as conventional CMC, hydroxypropyl methyl cellulose (HPMC), xanthan gum, and cross-linked carboxyvinyl polymers (e.g. carbomer).

U.S. Pat. No. 4,313,765 assigned to CP Kelco US Inc. discloses a composition comprising (a) 50 wt. % cellulase-free xanthan gum and (b) 50 wt. % carboxymethyl cellulose (CMC), hydroxyethyl cellulose (HEC) or hydroxypropyl methyl cellulose (HPMC) used as thickeners in formulations such as toothpaste, drink mix, etc.

U.S. Pat. No. 7,217,752 assigned to Lubrizol Advanced Materials Inc. discloses an aqueous composition containing a substantially crosslinked alkali-swellable acrylate copolymer rheology modifier, a surfactant, an alkaline material, and various compounds therein, as for example substantially insoluble materials requiring suspension or stabilization, such as a silicone, an oily material, or a pearlescent material.

PCT Publication 2019/025233 assigned to BASF SE, describes a cosmetic composition, especially hair care compositions, hair cleaning compositions or hair styling compositions, comprising a biobased polymer for improving the appearance and manageability of hair.

In view of the foregoing, there is a need for sufficiently stabilized aqueous surfactant-based compositions that include water-insoluble materials such as silicones, oily materials, pearlescent materials, cationic hair dyes, and other substantially insoluble materials.

Accordingly, it is an objective of the present application to provide a novel suspending system or composition which is free from or substantially free from carbomer for personal care and/or home care applications, wherein the suspending composition can produce sufficiently stabilized aqueous surfactant-based compositions that comprise at least one water insoluble material. Accordingly, the present application provides a unique blend consisting of naturally derived rheology modifiers to provide enhanced performance as compared to that of existing carbomer or acrylate-based rheology modifiers in surfactant-based personal care and/or home care applications.

Accordingly, the multifunctional natural based cellulosic/polysaccharide blend described in this application comprising hydroxypropylmethylcellulose (HPMC), xanthan gum and carboxymethyl cellulose (CMC) exhibits improved properties such as salt tolerance, compatibility with cationic ingredients, transparency, and does not require any neutralization. It also provides efficient suspension of water soluble ingredients in personal care and/or home care applications. Moreover, the composition is compatible with anionic, cationic, and nonionic ingredients. The natural based cellulosic/polysaccharide blend of this invention also demonstrates good electrolyte tolerance.

### SUMMARY OF THE INVENTION

The primary aspect of the present application is to provide a suspending polymer composition comprising a blend of: (a) about 50 wt. % to about 75 wt. % of hydroxypropylmethylcellulose (HPMC), (b) about 10 wt. % to about 25 wt. % of xanthan gum, and (c) about 10 wt. % to about 25 wt. % of carboxymethyl cellulose (CMC).

Another aspect of the present application is to provide a suspending polymer composition comprising a blend of: (a) about 65 wt. % of hydroxypropylmethylcellulose (HPMC), (b) about 17.5 wt. % to of xanthan gum, and (c) about 17.5 wt. % of carboxymethyl cellulose (CMC), wherein the carboxymethyl cellulose (CMC) has a degree of substitution (DS) of less than 0.65, or less than 0.60.

In another aspect, the suspending polymer composition of the present application is used in personal care compositions and/or in home care compositions.

In another aspect, the present application provides a personal care composition comprising: (a) about 0.05 wt. % to about 5 wt. % of a suspending polymer composition comprising a blend of: (i) about 50 wt. % to about 75 wt. % of hydroxypropylmethylcellulose (HPMC), (ii) about 10 wt. % to about 25 wt. % of xanthan gum, and (iii) about 10 wt. % to about 25 wt. % of carboxymethyl cellulose (CMC); (b) about 0.1 wt. % to about 30 wt. % of at least one surfactant selected from the group consisting of anionic, cationic, nonionic, zwitterionic, amphoteric and combinations thereof; (c) about 0.1 wt. % to about 10 wt. % of at least one personal care ingredient; and, (d) about 30 wt. % to about 70 wt. % water based on the total weight of the composition, wherein the composition is free from acrylic acids, methacrylic acids, or derivatives thereof.

In another aspect, the present application discloses a shampoo composition comprising: (a) about 0.05 wt. % to about 5 wt. % of a suspending polymer composition comprising a blend of: (i) about 50 wt. % to about 75 wt. % of hydroxypropylmethylcellulose (HPMC), (ii) about 10 wt. % to about 25 wt. % of xanthan gum, and (iii) about 10 wt. % to about 25 wt. % of carboxymethyl cellulose (CMC); (b) about 0.1 wt. % to about 30 wt. % of at least one surfactant selected from the group consisting of anionic, cationic, nonionic, zwitterionic, amphoteric and combinations thereof; (c) about 0.1 wt. % to about 10 wt. % of at least one shampoo ingredient; and, (d) about 30 wt. % to about 70 wt. % water based on the total weight of the composition, wherein the composition is free from acrylic acids, methacrylic acids, or derivatives thereof.

In another aspect, the present application discloses a home care composition comprising: (a) about 0.05 wt. % to about 5 wt. % of a suspending polymer composition comprising a blend of: (i) about 50 wt. % to about 75 wt. % of hydroxypropylmethylcellulose (HPMC), (ii) about 10 wt. % to about 25 wt. % of xanthan gum, and (iii) about 10 wt. % to about 25 wt. % of carboxymethyl cellulose (CMC); (b) about 0.1 wt. % to about 30 wt. % of at least one surfactant selected from the group consisting of anionic, cationic, nonionic, zwitterionic, amphoteric and combinations thereof; (c) about 0.1 wt. % to about 10 wt. % of at least one home care ingredient; and, (d) about 30 wt. % to about 70 wt. % water based on the total weight of the composition.

In yet another aspect, the present invention provides a fabric conditioner composition comprising: (a) about 0.05 wt. % to about 5 wt. % of a suspending polymer composition comprising a blend of: (i) about 50 wt. % to about 75 wt. % of hydroxypropylmethylcellulose (HPMC), (ii) about 10 wt. % to about 25 wt. % of xanthan gum, and (iii) about 10 wt. % to about 25 wt. % of carboxymethyl cellulose (CMC); (b) about 0.1 wt. % to about 30 wt. % of at least one surfactant selected from the group consisting of anionic, cationic, nonionic, zwitterionic, amphoteric and combinations thereof; (c) about 0.1 wt. % to about 10 wt. % of at least one fabric care ingredient; and, (d) about 30 wt. % to about 70 wt. % water based on the total weight of the composition.

### BRIEF DESCRIPTION OF THE FIGURES

Further embodiments of the present application can be understood with the appended figures.

FIG. 1 depicts the effect of accelerated stability test on shampoo compositions comprising suspending polymer compositions of Samples 1-3.

FIG. 2 depicts the effect of accelerated stability test on shampoo compositions comprising suspending polymer compositions of Sample 4 and Sample 6.

FIG. 3 depicts the effect of accelerated stability test on shampoo compositions comprising suspending polymer compositions of Samples 6-7.

FIG. 4 depicts the effect of accelerated stability test on shampoo compositions comprising suspending polymer compositions of Samples 14-16.

FIG. 5 depicts the effect of accelerated stability test on shampoo compositions comprising suspending polymer compositions of Samples 17-18.

FIG. 6 depicts the effect of accelerated stability test on anti-dandruff shampoo composition of Sample 19.

FIG. 7 depicts the effect of accelerated stability test on scentsual fabric conditioner composition of Sample 20.

FIG. 8 illustrates silicone deposition behavior of shampoo compositions of Samples 21-24 on Caucasian hair.

FIG. 9 illustrates silicone deposition behavior of shampoo compositions of Samples 23, and Samples 25-26 on Caucasian hair after subjected to one shampoo treatment.

FIG. 10 illustrates dry friction behavior of shampoo compositions of Samples 23-24 on Caucasian hair.

FIG. 11 illustrates impact of shampoo compositions of Samples 22-24 on foam stability.

FIG. 12 illustrates suspension stability of shampoo compositions of Samples 27-28 with Anton Paar Rheometer.

FIG. 13 illustrates performance of shampoo compositions of Sample 27 vs. Sample 29 across measuring shear range using Anton Paar Rheometer.

FIG. 14 illustrates performance of shampoo compositions of Sample 28 vs. Sample 29 across measuring shear range using Anton Paar Rheometer.

FIG. 15 depicts temperature stability of sulfate-free shampoo compositions of Sample 27 vs. Sample 29.

FIG. 16 illustrates large amplitude oscillation shear (LAOS) measurement curves indicating the product texture of sulfate-free shampoo compositions of Sample 27 vs. Sample 29.

FIG. 17 illustrates large amplitude oscillation shear (LAOS) measurement curves indicating the product texture of sulfate-free shampoo compositions of Sample 28 vs. Sample 29.

FIG. 18 depicts images indicating the product texture of sulfate-free shampoo compositions of Sample 27 vs. Sample 29.

FIG. 19 illustrates flow profiles indicating the thickening effect of sulfate-free shampoo compositions of Sample 27-29 measured using Anton Paar Rheometer.

FIG. 20 illustrates synergistic effect on the viscosity of the shampoo composition of Example-5.

FIG. 21 illustrates anti-dandruff agent deposition behavior of anti-dandruff shampoo compositions of Sample 30 vs. Sample 31.

### DETAILED DESCRIPTION OF THE INVENTION

Before explaining at least one aspect of the disclosed and/or claimed inventive concept(s) in detail, it is to be understood that the disclosed and/or claimed inventive concept(s) is not limited in its application to the details of construction and the arrangement of the components or steps or methodologies set forth in the following description or illustrated in the drawings. The disclosed and/or claimed inventive concept(s) is capable of other aspects or of being practiced or carried out in various ways. Also, it is to be understood that the phraseology and terminology employed herein is for the purpose of description and should not be regarded as limiting.

As utilized in accordance with the disclosure, the following terms, unless otherwise indicated, shall be understood to have the following meanings.

Unless otherwise defined herein, technical terms used in connection with the disclosed and/or claimed inventive concept(s) shall have the meanings that are commonly understood by those of ordinary skill in the art. Further, unless otherwise required by context, singular terms shall include pluralities and plural terms shall include the singular.

The singular forms “a,” “an,” and “the” include plural forms unless the context clearly dictates otherwise specified or clearly implied to the contrary by the context in which the reference is made. The term “Comprising” and “Comprises of” includes the more restrictive claims such as “Consisting essentially of” and “Consisting of”.

For purposes of the following detailed description, other than in any operating examples, or where otherwise indicated, numbers that express, for example, quantities of ingredients used in the specification and claims are to be understood as being modified in all instances by the term “about”. The numerical parameters set forth in the specification and attached claims are approximations that may vary depending upon the desired properties to be obtained in carrying out the invention.

All percentages, parts, proportions, and ratios as used herein, are by weight of the total composition, unless otherwise specified. All such weights as they pertain to listed ingredients are based on the active level and, therefore; do not include solvents or by-products that may be included in commercially available materials, unless otherwise specified.

All publications, articles, papers, patents, patent publications, and other references cited herein are hereby incorporated herein in their entirety for all purposes to the extent consistent with the disclosure herein.

The use of the term “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more depending on the term to which it is attached. In addition, the quantities of 100/1000 are not to be considered limiting as lower or higher limits may also produce satisfactory results.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “each independently selected from the group consisting of” means when a group appears more than once in a structure, that group may be selected independently each time it appears.

The term “polymer” refers to a compound comprising repeating structural units (monomers) connected by covalent chemical bonds. Polymers may be further derivatized, crosslinked, grafted or end-capped. Non-limiting examples of polymers include copolymers, terpolymers, tetrapolymers, quaternary polymers, and homologues. The term “copolymer” refers to a polymer consisting essentially of two or more different types of monomers polymerized to obtain the copolymer.

In a non-limiting embodiment, the present application discloses a suspending polymer composition comprising a blend of: (a) about 50 wt. % to about 75 wt. % of hydroxypropylmethylcellulose (HPMC), (b) about 10 wt. % to about 25 wt. % of xanthan gum, and (c) about 10 wt. % to about 25 wt. % of carboxymethyl cellulose (CMC).

As used herein, “hydroxypropylmethylcellulose (HPMC)” refers to the following non-limiting commercially available HPMC products under the tradenames as Methocel® from Dow, PharmaCoat® from Shin-Etsu, and Benecel® or Culminal MHPC from Ashland LLC.

As used herein, ‘xanthan gum’ refers to a polysaccharide gum produced by the Xanthomonas campestris bacterium. Xanthan gum is useful industrially capable of producing a large increase in the viscosity of a liquid. Any commercially available xanthan gum may be used in the present invention. The suitable commercial xanthan gum include, but are not limited to, Xanthan Gum®, (Kelzan® from Kelco), Rhodopol®23 (Rhone Poulenc) or Veegum® (from R. T. Vanderbilt). The preferred high molecular weight polysaccharides of the present invention are xanthan gum having a molecular weight of about 10,000 to 600,000 Daltons.

As used herein, carboxymethyl cellulose (CMC) refers to the non-limiting commercially available CMC products sold under AQUASORB series of trade designations including e.g., AQUASORB A380 and AQUASORB A500, available from Ashland LLC, Akucell& trade designations including e.g., Akucell® AF 3285, available from Nouryon, or other suitable grades of carboxymethyl cellulose (CMC) commercially available in the market.

In some embodiments, the suitable range of hydroxypropylmethylcellulose (HPMC) for the present application can be varied from about 40 wt. % to about 45 wt. %, or from about 45 wt. % to about 50 wt. %, or from about 50 wt. % to about 55 wt. %, or from about 55 wt. % to about 60 wt. %, or from about 60 wt. % to about 65 wt. %, or from about 65 wt. % to about 70 wt. %, or from about 70 wt. % to about 75 wt. %, or from about 75 wt. % to about 80 wt. %, based on the total weight of the suspending polymer composition

In some embodiments, the suitable range of xanthan gum for the present application can be varied from about 5 wt. % to about 10 wt. %, or from about 10 wt. % to about 15 wt. %, or from about 15 wt. % to about 20 wt. %, or from about 20 wt. % to about 25 wt. %, or from about 25 wt. % to about 30 wt. %, based on the total weight of the suspending polymer composition.

In some embodiments, the suitable range of carboxymethyl cellulose (CMC) for the present application can be varied from about 5 wt. % to about 10 wt. %, or from about 10 wt. % to about 15 wt. %, or from about 15 wt. % to about 20 wt. %, or from about 20 wt. % to about 25 wt. %, or from about 25 wt. % to about 30 wt. %, based on the total weight of the suspending polymer composition.

In some embodiments, the carboxymethyl cellulose (CMC) for the present application has a degree of substitution (DS) of less than 0.65, or less than 0.60.

In a non-limiting embodiment, the present application discloses a suspending polymer composition that can be used in personal care and/or home care compositions, wherein, the compositions can be aqueous or non-aqueous based compositions.

In a non-limiting embodiment of the present application a personal care composition comprises: (a) about 0.05 wt. % to about 5 wt. % of a suspending polymer composition comprising a blend of: (i) about 50 wt. % to about 75 wt. % of hydroxypropylmethylcellulose (HPMC), (ii) about 10 wt. % to about 25 wt. % of xanthan gum, and (iii) about 10 wt. % to about 25 wt. % of carboxymethyl cellulose (CMC); (b) about 0.1 wt. % to about 30 wt. % of at least one surfactant selected from the group consisting of anionic, cationic, nonionic, zwitterionic, amphoteric and combinations thereof; (c) about 0.1 wt. % to about 10 wt. % of at least one personal care ingredient; and, (d) about 30 wt. % to about 70 wt. % water based on the total weight of the composition, wherein the composition is free from acrylic acids, methacrylic acids, or derivatives thereof.

In an another non-limiting embodiment of the present application discloses a home care composition comprises: (a) about 0.05 wt. % to about 5 wt. % of a suspending polymer composition comprising a blend of: (i) about 50 wt. % to about 75 wt. % of hydroxypropylmethylcellulose (HPMC), (ii) about 10 wt. % to about 25 wt. % of xanthan gum, and (iii) about 10 wt. % to about 25 wt. % of carboxymethyl cellulose (CMC); (b) about 0.1 wt. % to about 30 wt. % of at least one surfactant selected from the group consisting of anionic, cationic, nonionic, zwitterionic, amphoteric and combinations thereof; (c) about 0.1 wt. % to about 10 wt. % of at least one home care ingredient; and, (d) about 30 wt. % to about 70 wt. % water based on the total weight of the composition.

The suitable surfactant for the purpose of the present application is selected from the group consisting of anionic, zwitterionic, amphoteric, nonionic, or cationic surfactant, or combinations thereof.

Nonionic surfactants can be broadly defined as compounds containing a hydrophobic moiety and a nonionic hydrophilic moiety. Examples of the hydrophobic moiety can be alkyl, alkyl aromatic, dialkyl siloxane, polyoxyalkylene, and fluoro-substituted alkyls. Examples of hydrophilic moieties are polyoxyalkylenes, phosphine oxides, sulfoxides, amine oxides, and amides. Nonionic surfactants such as those marketed under the trade name Surfynol® are also useful in this invention. Nonionic surfactants useful herein include compounds produced by the condensation of alkylene oxide groups (hydrophilic in nature) with an organic hydrophobic compound which maybe aliphatic or alkylaromatic in nature. Non-limiting examples of suitable nonionic surfactants include: poloxamers (sold under the trade name Pluronic® by BASF Corporation), polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides, and blends thereof.

Cationic surfactants useful in the compositions of the present invention, may contain amino or quaternary ammonium hydrophilic moieties which are positively charged when dissolved in the aqueous composition of the present invention. Non-limiting examples of suitable cationic surfactants include derivatives of aliphatic quaternary ammonium compounds having at least one long alkyl chain containing from about 8 to about 18 carbon atoms, such as, lauryl trimethylammonium chloride, cetyl pyridinium chloride, cetyl trimethylammonium bromide, di-isobutylphenoxyethyl-dimethylbenzylammonium chloride, coconut alkyltrimethylammomum nitrite, cetyl pyridinium fluoride, and blends thereof. Further suitable are quaternary ammonium fluorides having detergent properties such as compounds described in U.S. Pat. No. 3,535,421.

Cationic surfactants can have a hydrophobe that carries a positive charge or that is uncharged at pH values close to neutrality or lower, such as alkylamines, alkyl imidazolines, ethoxylated amines, and quaternary ammonium compounds. Alkylamines can be salts of primary, secondary and tertiary fatty C₁₂-C₂₂ alkylamines, substituted or unsubstituted, and substances sometimes referred to as “amidoamines”. Non-limiting examples of alkylamines and salts thereof include dimethyl cocamine, dimethyl palmitamine, dioctylamine, dimethyl stearamine, dimethyl soyamine, soyamine, myristyl amine, tridecyl amine, ethyl stearylamine, N-tallowpropane diamine, ethoxylated stearylamine, dihydroxy ethyl stearylamine, arachidylbehenylamine, dimethyl lauramine, stearylamine hydrochloride, soyamine chloride, stearylamine formate, N-tallowpropane diamine dichloride, and amodimethicone (INCI name for a silicone polymer and blocked with amino functional groups, such as aminoethylamino propylsiloxane). Non-limiting examples of amidoamines and salts thereof include stearamido propyl dimethyl amine, stearamidopropyl dimethylamine citrate, palmitamidopropyl diethylamine, and cocamidopropyl dimethylamine lactate. Other cationic surfactants include distearyldimonium chloride, dicetyldimonium chloride, guar hydroxypropyltrimonium chloride, and the like. At low pH, amine oxides may protonate and behave similarly to N-alkyl amines. Non-limiting examples of alkyl imidazolines include alkyl hydroxyethyl imidazoline, such as stearyl hydroxyethyl imidazoline, coco hydroxyethyl imidazoline, ethyl hydroxymethyl oleyl oxazoline, and the like. Non-limiting examples of ethyoxylated amines include PEG-cocopolyamine, PEG-15 tallow amine, quaternium-52, and the like.

Quaternary ammonium compounds can be selected from monomeric or polymeric materials containing at least one nitrogen atom that is linked covalently to four alkyl and/or aryl substituents, and the nitrogen atom remains positively charged regardless of the environmental pH. Quaternary ammonium compounds comprise a large number of substances that are used extensively as surfactants, conditioners, antistatic agents, and antimicrobial agents and include, alkylbenzyldimethyl ammonium salts, alkyl betaines, heterocyclic ammonium salts, and tetraalkylammonium salts. Long-chain (fatty) alkylbenzyldimethyl ammonium salts are preferred as conditioners, as antistatic agents, and as fabric softeners, discussed in more detail below. Other quaternary ammonium compounds include quaternary ammonium silicones. While various quaternary ammonium compounds are listed for a specific purpose, one of ordinary skill will recognize that the quaternary ammonium compounds described here and throughout the specification can serve more than one function.

Non-limiting examples of alkylbenzyldimethylammonium salts include stearalkonium chloride, benzalkonium chloride, quaternium-63, olealkonium chloride, didecyldimonium chloride, and the like. Alkyl betaine compounds include alkylamidopropyl betaine, alkylamidopropyl hydroxysultaine, and sodium alkylamido propyl hydroxyphostaine. Non-limiting examples of alkyl betaine compounds include oleyl betaine, coco-betaine, cocoamidopropyl betaine, coco-hydroxy sultaine, coco/oleamidopropyl betaine, coco-sultaine, cocoamidopropylhydroxy sultaine, and sodium lauramidopropyl hydroxyphostaine. Heterocyclic ammonium salts include alkylethyl morpholinium ethosulfate, isostearyl ethylimidonium ethosulfate, and alkylpyridinium chlorides, and are generally used as emulsifying agents. Non-limiting examples of heterocyclic ammonium salts include cetylpyridinium chloride, isostearylethylimidonium ethosulfate, and the like. Non-limiting examples of tetraalkylammonium salts include cocamidopropyl ethyldimonium ethosulfate, hydroxyethyl cetyldimonium chloride, quaternium-18, and cocodimonium hyroxypropyl hydrolyzed protein, such as hair keratin, and the like.

Zwitterionic surfactants are exemplified by those which can be broadly described as derivative of aliphatic quaternary ammonium, phosphonium, and sulfonium compounds, which can be broadly described as derivative of aliphatic quaternary ammonium, phosphonium, and sulfonium compounds, in which the aliphatic radicals can be straight or branched chain, and wherein one of the aliphatic substituents contains from about 8 to about 18 carbon atoms and one contains as anionic water-solubilizing group, e.g., carboxy, sulfonate, sulfate, phosphate, or phosphonate. Non-limiting examples of suitable zwitterionic surfactants include betaines and derivatives of aliphatic quaternary ammonium compounds in which the aliphatic radicals can be straight chain or branched, and which contain an anionic water-solubilizing group, e.g., carboxy, sulfonate, sulfate, phosphate, or phosphonate.

Examples of amphoteric surfactants which can be used in the vehicle systems of the compositions of the present invention are those which are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate.

Anionic surfactants useful herein include the water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate (SLS) and sodium coconut monoglyceride sulfonates are non-limiting examples of anionic surfactants of this type. Non-limiting examples of suitable anionic surfactants include: sarcosinates, taurates, isethionates, sodium lauryl sulfoacetate, sodium laureth carboxylate, and sodium dodecyl benzenesulfonate. Also suitable are alkali metal or ammonium salts of surfactants such as the sodium and potassium salts of the following: lauroyl sarcosinate, myristoyl sarcosinate, palmitoyl sarcosinate, stearoyl sarcosinate, and oleoyl sarcosinate. Other surfactants such as fluorinated surfactants may also be incorporated within the compositions of the invention.

Anionic surfactants include substances having a negatively charged hydrophobe or that carry a negative charge when the pH is elevated to neutrality or above, such as acylamino acids, and salts thereof, for example, acylglutamates, acyl peptides, sarcosinates, and taurates; carboxylic acids, and salts thereof, for example, alkanolic acids and alkanoates, ester carboxylic acids, and ether carboxylic acids; phosphoric acid ester and salts thereof; sulfonic acids and salts thereof, for example, acyl isethionates, alkylaryl sulfonates, alkyl sulfonates, and sulfosuccinates; and sulfuric acid esters, such as alkyl ether sulfates and alkyl sulfates.

Anionic surfactants useful herein include the water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate (SLS) and sodium coconut monoglyceride sulfonates are non-limiting examples of anionic surfactants of this type. Non-limiting examples of suitable anionic surfactants include: sarcosinates, taurates, isethionates, sodium lauryl sulfoacetate, sodium laureth carboxylate, and sodium dodecyl benzenesulfonate. Also suitable are alkali metal or ammonium salts of surfactants such as the sodium and potassium salts of the following: lauroyl sarcosinate, myristoyl sarcosinate, palmitoyl sarcosinate, stearoyl sarcosinate, and oleoyl sarcosinate. Other surfactants such as fluorinated surfactants may also be incorporated within the compositions of the invention.

Non-limiting examples of anionic surfactants include mono-basic salts of acylglutamates that are slightly acidic in aqueous solution, such as sodium acylglutamate and sodium hydrogenated tallow glutamate; salts of acyl-hydrolyzed protein, such as potassium, palmitoyl hydrolyzed milk protein, sodium cocoyl hydrolyzed soy protein, and TEA-abietoyl hydrolyzed collagen; salts of acyl sarcosinates, such as ammonium myristoyl sarcosine, sodium cocoyl sarcosinate, and TEA-lauroyl sarcosinate; salts of sodium methyl acyltaurates, such as sodium lauroyl taurate and sodium methyl cocoyl taurate; alkanoic acids and alkanoates, such as fatty acids derived from animal and vegetable glycerides that form water-soluble soaps and water-insoluble emulsifying soaps, including sodium stearate, aluminum stearate, and zinc undecylenate; ester carboxylic acids, such as dinonoxynol-9-citrate; salts of acyl lactylates such as calcium stearoyl lactylate and laureth-6 citrate; ethercarboxylic acids derived from ethyoxylated alcohols or phenols having varying lengths of polyoxyethylene chains, such as nonoxynol-8 carboxylic acid, and sodium trideceth-13 carboxylate; mono- and di-esters of phosphoric acid and their salts, such as phospholipids, dilaureth-4-phosphate, DEA-oleth-10 phosphate and triethanolamine lauryl phosphate; salts of acylisethionate, such as sodium cocoyl isethionate; alkylarylbenzene sulfonates, such as alpha-olefin sulfonate (AOS) and alkali metal, alkaline earth metal, and alkanolamine salts thereof, and sodium dodecylbenzene sulfonate; alkyl sulfonates, s such as sodium C₁₂-C₁₄ olefin sulfonate, sodium cocomonoglyceride sulfonate, sodium C₁₂-C₁₅ pareth-15 sulfonate, and sodium lauryl sulfoacetate; sulfosuccinates, such as mono- and di-esters of sulfosuccinic acid, salts thereof and alkoxylated alkyl and alkylamido derivatives thereof, such as di-C₄-C₁₀ alkyl sodium sulfosuccinate, disodium laureth sulfosuccinate, disodium oleamido MEA-sulfosuccinate, and disodium C₁₂-C₁₅ pareth sulfosuccinate; alkyl ether sulfates, such as sodium and ammonium lauryl ether sulfate (having about 1 to about 12 moles ethylene oxide); alkyl sulfates, such as sodium, ammonium and triethanolamine salts of C₁₂-C₁₈ alkylsulfates, sodium C₁₂-C₁₄ olefin sulfates, sodium laureth-6 carboxylate, sodium C₁₂-C₁₈ pareth sulfate, and the like.

Also suitable surfactants are selected from the following commercial products: (1) Alkanolamides, under the trade names Amidex and Schercomid; amido-amines, under the trade names Katemul and Schercodine; amine oxides, under the trade names Chemoxide™ and Schercamox™; amphoterics, under the trade names Chembetaine™, Schercotaine™ and Schercoteric™; imidazolines, under the trade name Schercozoline™; pearlizing agents, under the trade name Quickpearl™; performance concentrates, under the trade names Sulfochem™ and Chemoryl™; soaps (potassium cocoate and potassium soyate); specialty ethoxylates, under the trade name Chemonic™; specialty quats under the trade names Quatrex and Schercoquat, sulfates, under the trade name Sulfochem; and sulfosuccinates, under the trade name Chemccinate™ from Lubrizol. (2) Avaniel, Cremaphore® Jordapan® and Pluracare® from BASF Corp. (3) Miracare® SLB, Mackam® Bab, Mackanate® Ultra SI, Miranof Ultra, and Miracare® Plaisant from Rhodia.] (4) Stepan® Pearl 2, Stepan® Pearl 4, Stepan® Pearl Series, Neobee® M-20, Stepan® PTC, Amphosol® 2CSF, Steol® Stepan-Mild® GCC, StepanR SLI\hat{ }FB, Stepanol® AM, Stepanol® PB, Alpha-Step® BSS-45, Bio-Terge® 804, Stepan-MildR L3, Stepan® SLL-FB, Stepan® SSL-CG, and Stepanol® CFAS-70 from Stepan Company.

Also, suitable surfactants can be selected from those described in the book Surfactants in Personal Care Products and Decorative Cosmetics, Third Edition, 2006, CRC Press, the disclosure is incorporated hereby in its entirety by reference.

According to another embodiment, the present application contemplates use of an anionic surfactant selected from the group consisting of alkyl sulfates, alkyl ether sulfates and combinations thereof; a nonionic surfactant selected from the group consisting of C₆-12 alcohol alkoxylates, seed oil alcohol alkoxylates, and combinations thereof; a zwitterionic surfactant selected from the group consisting of betaines such as, cocamidoalkyl betaine, cocamidopropyl betaine, and combinations thereof.

In some embodiments, the present application discloses suitable range of surfactant that can be varied from about 0.1 wt. % to about 1 wt. %, or from about 1 wt. % to about 2.5 wt. %, or from about 2.5 wt. % to about 5 wt. % or from about 5 wt. % to about 10 wt. %, or 10 wt. % to about 15 wt. %, or from about 15 wt. % to about 20 wt. %, or from about 20 wt. % to about 25 wt. %, or from about 25 wt. % to about 30 wt. % based on the total weight of the personal care and/or home care compositions.

According to another embodiment, the present application discloses a shampoo composition comprises: (a) about 0.05 wt. % to about 5 wt. % of a suspending polymer composition comprising a blend of: (i) about 50 wt. % to about 75 wt. % of hydroxypropylmethylcellulose (HPMC), (ii) about 10 wt. % to about 25 wt. % of xanthan gum, and (iii) about 10 wt. % to about 25 wt. % of carboxymethyl cellulose (CMC); (b) about 0.1 wt. % to about 30 wt. % of at least one surfactant selected from the group consisting of anionic, cationic, nonionic, zwitterionic, amphoteric and combinations thereof; (c) about 0.1 wt. % to about 10 wt. % of at least one shampoo ingredient; and, (d) about 30 wt. % to about 70 wt. % water based on the total weight of the composition, wherein the composition is free from acrylic acids, methacrylic acids, or derivatives thereof.

According to another embodiment, the shampoo composition of the present application is a 2-in-1 shampoo composition, a 3-in-1 shampoo composition, a sulfate free shampoo composition, a silicone-free shampoo composition, an antidandruff shampoo composition, or a natural shampoo composition.

According to another embodiment, the present application discloses a fabric conditioner composition comprising: (a) about 0.05 wt. % to about 5 wt. % of a suspending polymer composition comprising a blend of: (i) about 50 wt. % to about 75 wt. % of hydroxypropylmethylcellulose (HPMC), (ii) about 10 wt. % to about 25 wt. % of xanthan gum, and (iii) about 10 wt. % to about 25 wt. % of carboxymethyl cellulose (CMC); (b) about 0.1 wt. % to about 30 wt. % of at least one surfactant selected from the group consisting of anionic, cationic, nonionic, zwitterionic, amphoteric and combinations thereof; (c) about 0.1 wt. % to about 10 wt. % of at least one fabric care ingredient; and, (d) about 30 wt. % to about 70 wt. % water based on the total weight of the composition.

According to another embodiment of the present application, it is contemplated to employ at least one personal care ingredient selected from the group consisting of water-insoluble ingredients, oxidizing agents, conditioning agents, humectants, pH adjusting buffers, waxes, mineral oils, emulsifiers, fragrances, preservatives, fatty substances, gelling agents, thickeners, moisturizers, emollients, hydrophilic or lipophilic active agent, antioxidants, sequestering agents, acidifying or basifying agents, fillers, dyestuffs, plant extracts, proteins, peptides, neutralizing agents, solvents, anti-dandruff ingredients, reducing agents and combinations thereof.

In some embodiments, the suitable range of personal care or home care ingredient for the present application can be varied from about 0.1 wt. % to about 1 wt. %, or from about 1 wt. % to about 5 wt. %, or from about 5 wt. % to about 10 wt. % based on the total weight of the personal care or home care composition.

According to another embodiment of the present application, it is contemplated to employ at least one home care ingredient selected from the group consisting of water-insoluble ingredients, rheology modifiers, solvents, phosphates, phosphonates, chelating agents, defoamers, anti-redeposition agents, fabric conditioners, fabric softeners, polish additives, plasticizers, colorants, preservatives, fragrances, microencapsulated fragrances, antifoam compounds, antibacterial or antifungal agents, abrasives, enzymes, optical brighteners, antioxidants, bleaching agents, emulsifiers, functional polymers, viscosifying agents, alcohols, oils, dye transfer inhibitors, hydrotropes, dye sequestrants, color fixatives, pH controlling agents, electrolytes, and combinations thereof.

In some embodiments, the suitable range of home care ingredient for the present application can be varied from about 0.1 wt. % to about 1 wt. %, or from about 1 wt. % to about 5 wt. %, or from about 5 wt. % to about 10 wt. % based on the total weight of the personal care or home care composition.

According to another embodiment of the present application, it is contemplated to employ at least one shampoo ingredient selected from the group consisting of water-insoluble ingredients, oxidizing agents, conditioning agents, humectants, pH adjusting buffers, waxes, mineral oils, emulsifiers, fragrances, preservatives, fatty substances, gelling agents, thickeners, moisturizers, emollients, hydrophilic or lipophilic active agent, antioxidants, sequestering agents, acidifying or basifying agents, fillers, dyestuffs, plant extracts, proteins, peptides, neutralizing agents, solvents, anti-dandruff ingredients, reducing agents and combinations thereof.

In some embodiments, the suitable range of shampoo ingredient for the present application can be varied from about 0.1 wt. % to about 1 wt. %, or from about 1 wt. % to about 5 wt. %, or from about 5 wt. % to about 10 wt. % based on the total weight of the personal care or home care composition.

According to another embodiment of the present application, it is contemplated to employ at least one fabric care ingredient selected from the group consisting of water-insoluble ingredients, rheology modifiers, solvents, phosphates, phosphonates, chelating agents, defoamers, anti-redeposition agents, fabric conditioners, fabric softeners, polish additives, plasticizers, colorants, preservatives, fragrances, microencapsulated fragrances, antifoam compounds, antibacterial or antifungal agents, abrasives, enzymes, optical brighteners, antioxidants, bleaching agents, emulsifiers, functional polymers, viscosifying agents, alcohols, oils, dye transfer inhibitors, hydrotropes, dye sequestrants, color fixatives, pH controlling agents, electrolytes, and combinations thereof.

In some embodiments, the suitable range of fabric care ingredient for the present application can be varied from about 0.1 wt. % to about 1 wt. %, or from about 1 wt. % to about 5 wt. %, or from about 5 wt. % to about 10 wt. % based on the total weight of the personal care or home care composition.

According to the present application, the suitable range of water-insoluble ingredient present in the home care or personal care composition can be varied from about 0.1 wt. % to about 1 wt. %, or from about 1 wt. % to about 2.5 wt. %, or from about 2.5 wt. % to about 5 wt. % or from about 5 wt. % to about 10 wt. %, based on the total weight of the personal care or home care composition.

According to the present application, non-limiting examples of the water-insoluble ingredient is selected from the group consisting of silicones, natural and synthetic oils, emollients, organic and/or inorganic pearlescent pigments, beads, fatty alcohols, discrete particles, polymers, and combinations thereof.

According to the present application, non-limiting examples of silicones are selected from the group consisting of polydimethylsiloxane, amodimethicone, amodimethicone macroemulsion or microemulsion, dimethicone, dimethiconol (silicone gum), dimethiconol microemulsion or macroemulsion, cyclomethicone, phenyltrimethicone, organo polysiloxane, alkoxysilicone, or any combination thereof. The silicones used herein, can be selected from silicone fluids, silicone oils, cationic silicones, silicone gums, high refractive silicones, silicone resins, emulsified silicones, dimethicone copolyols, and combinations thereof.

Dimethicone copolyols used herein, are disclosed in U.S. Pat. Nos. 5,136,063 and 5,180,843, the disclosures of which are incorporated herein by reference. In addition, dimethicone copolyols are commercially available under the Silsoft® and Silwet® brand names from the General Electric Company (GE-OSi). Specific product designations include but are not limited to Silsoft 305, 430, 475, 810, 895, Silwet L 7604 (GE-OSi); Dow Corning® 5103 and 5329 from Dow Corning Corporation; and Abil® dimethicone copolyols, such as, for example WE 09, WS 08, EM 90 and EM 97 from Evonik Goldschmidt Corporation; and Silsense™ dimethicone copolyols, such as Silsense Copolyol-1 and Silsense Copolyol-7, available from Lubrizol Advanced Materials, Inc.

In another non-limiting embodiment, the water-insoluble pearlescent pigments are selected from the group consisting of titanium dioxide, zirconium oxide or cerium oxide, zinc oxide, iron oxide or chromium oxide, manganese violet, ultramarine blue and ferric blue, carbon black, and lakes based on cochineal carmine or on barium, strontium, calcium or aluminium, white nacreous pigments such as mica coated with titanium or with bismuth oxychloride, colored nacreous pigments such as titanium mica with iron oxides, titanium mica especially with ferric blue or with chromium oxide and mixtures thereof.

In a non-limiting embodiment, the present application discloses a shampoo composition that includes particles of titanium dioxide coated mica. These particles may vary in size from 2 to 150 μm in diameter. In general, smaller particles give rise to a shampoo composition having a pearly appearance, whereas particles having a larger average diameter will result in a glittery shampoo composition.

According to another embodiment of the present application, it is contemplated to employ at least one discrete particle selected from the group consisting of scrubbing agents, captivates such as microencapsulated fragrances, and mixtures thereof.

According to another embodiment of the present application, it is contemplated to employ at least one emollient selected from the group consisting of oil soluble bio-functionals, silicone oils, silicone derivatives, essential oils, oils, fats, fatty acids, fatty acid esters, fatty alcohols, waxes, polyols, hydrocarbons, and mixtures thereof.

In a non-limiting embodiment, present application discloses the w/w % of the suspending polymer composition in a personal care and/or home care compositions ranges from about 0.05 wt. % to about 0.1 wt. %, from about 0.1 wt. % to about 1 wt. %, or from about 1 wt. % to about 2.5 wt. %, or from about 2.5 wt. % to about 5 wt. % according to type of personal care and/or home care compositions.

As per another embodiment of the present application, the personal care and/or home care composition is formulated into a spray, a lotion, a mousse, a fluid, a serum, a solution, a perm, an emulsion, a gel, a vesicle a dispersion, a paste, a cream, a solid stick, a shampoo, a balm, a wipe, a milk, a foam or a jelly.

According to another non-limiting embodiment of the present application, the personal care and/or home care compositions can be a rinse-off type compositions, wherein such rinse-off type products comprising the suspending polymer composition provide desired suspension of water insoluble ingredients.

According to another non-limiting embodiment of the present application, the personal care and/or home care compositions can be a leave-on type compositions, wherein such leave-on type products comprising the suspending polymer composition provide desired suspension of water insoluble ingredients.

In another embodiment, it is contemplated that the suspending polymer composition can be used to thicken the water based systems or compositions irrespective of the application, particularly, personal care compositions, home care compositions, industrial compositions, coating compositions, ink compositions, metal fluid compositions, cement compositions, oil well drilling fluid compositions, oral care compositions, food compositions, and beverage compositions.

In another nonlimiting embodiment of the present application, the personal care composition can be an appropriate product selected from the group consisting of hair-care products, shampoos, hair conditioners, 2-in-1 shampoos, 3-in-1 shampoos, sulfate free shampoos, silicone-free shampoos, antidandruff shampoos, leave in and rinse off conditioners, hair treatments including intensive treatments, styling and treating hair compositions, hair perming products, hair straighteners, hair relaxants, hair sprays and lacquers, permanent hair dyeing systems, hair styling mousses, hair gels, semipermanent hair dyeing systems, temporary hair dyeing systems, hair bleaching agents, permanent hair wave systems, hair setting formulations, non-coloring hair preparations, hair-frizz-control gels, hair leave-in conditioners, hair pomades, hair de-tangling products, hair fixatives, hair conditioning mists, hair care pump sprays and other non-aerosol sprays, skin-care products, hair cuticle coats, skin care moisturizing mists, skin wipes, pore skin wipes, pore cleaners, blemish reducers, skin exfoliators, skin desquamation enhancers, skin towelettes, skin protection ointments, skin powders, skin pads, paste masks and muds, face masks, facial cleansing products, anti-acne preparations, bath products, shower products, liquid soaps, bar soaps, body oils, body lotions, body gels, body and hand preparations, face and body washes, bath salts, bath and body milks, foam baths, synthetic and non-synthetic soap bars, hand liquids, shaving lotions, shaving and aftershave preparations, pre-shaves and pre-electric shaves, nail varnishes, nail polish, nail polish remover, nail creams and lotions, cuticle softeners, nail conditioners, eye shadows, mascaras, eye liners, eye shadows, blushes, makeup, eye shadow sticks, baby lotions, baby baths and shampoos, baby conditioners, fragrances and/or odoriferous ingredients consisting preparations, dentifrices, deodorizing and antiperspirant preparations, decorative preparations, light protection formulations, treatment creams, lipsticks, dry and moist make-up, rouge, powders, depilatory agents, sun care products, compositions comprising UV blockers or UV protectors, anti-aging products, foundations, face powders, moisturizing preparations, tanning preparations, nose strips, make-up removers, cold creams, mousses, shower gels, personal care rinse-off products, gels, scrubbing cleansers, astringents, lip balms, lip glosses, anhydrous creams and lotions, oil/water, water/oil, multiple and macro and micro emulsions, water-resistant creams and lotions, mouth-washes, massage oils, toothpastes, clear gels and sticks, ointment bases, topical wound-healing products, aerosol talc, barrier sprays, vitamin, herbal-extract preparations, and/or controlled-release personal care products.

In another embodiment of the present application, it is contemplated that the personal care compositions comprising the suspending polymer composition of the present invention can be selected from the group consisting of hair-care products, shampoos, hair conditioners, leave in and rinse off conditioners, styling and treating hair compositions, hair perming products, hair relaxants, hair straighteners, hair sprays and lacquers, permanent hair dyeing systems, hair styling mousses, hair gels, semi-permanent hair dyeing systems, temporary hair dyeing systems, hair bleaching systems, permanent hair wave systems, hair setting formulations, skin-care products, bath products, shower products, liquid soaps, bar soaps, fragrances and/or odoriferous ingredients consisting preparations, deodorizing and antiperspirant preparations, decorative preparations, light protection formulations, shaving lotions, body oils, body lotions, body gels, treatment creams, body cleaning products, skin protection ointments, shaving and aftershave preparations, skin powders, lipsticks, nail varnishes, eye shadows, mascaras, dry and moist make-up, rouge, powders, depilatory agents and sun care products.

According to another embodiment of the present application, it is contemplated that the home care compositions comprising the suspending polymer composition of the present invention can be selected from the group consisting of laundry washing products, dish washing products, fabric conditioner, heavy duty cleaning products, disinfecting products, fabric enhancing products, fabric softener, fabric abrasion reducing products, toilet cleaning products, floor cleaning products, window cleaning products, auto polishing products, automobile cleaning products, shoe polishing products, household fragrance products, wall coloring products and wall paper adhesives.

According to yet another embodiment the present application, the suspending polymer composition is used in personal care compositions wherein the personal care compositions is completely free from or substantially free from acrylic acids, methacrylic acids, or derivatives thereof.

Further, certain aspects of the present application are illustrated in detail by way of the following examples. The examples are given herein for illustration of the application and are not intended to be limiting thereof.

### EXAMPLES

### Example-1: Preparation of Suspending Polymer Compositions

Hydroxypropylmethylcellulose (HPMC), xanthan gum, and carboxymethyl cellulose (CMC) were weighed in a bottle according to their respective w/w ratios as given in Table-1. The mixture of ingredients in powder form was then subjected to homogenization in IKA ROLLER 6 apparatus for 30 minutes at a speed of 80 rpm. Homogenized blends of Samples 1-13 were obtained using above method.

TABLE 1
suspending polymer compositions.

| | HPMC | CMC DS < 0.6 | Xanthan gum | CMC DS < 0.6 | |
|---|---|---|---|---|---|
| Samples | (% w/w) | 75 mesh (% w/w) | (% w/w) | 200 mesh (% w/w) | Stability |
| Sample 1 | — | 100 | — | — | Unstable |
| Sample 2 | — | — | 100 | — | Unstable |
| Sample 3 | 100 | — | — | — | Unstable |
| Sample 4 | — | 50 | 50 | — | Unstable |
| Sample 5 | 50 | — | 50 | — | Unstable |
| Sample 6 | 50 | 50 | — | — | Unstable |
| Sample 7 | 65 | 17.5 | 17.5 | — | Stable |
| Sample 8 | 50 | 25 | 25 | — | Stable |
| Sample 9 | 70 | 15 | 15 | — | Stable |
| Sample 10 | 60 | 25 | 15 | — | Stable |
| Sample 11 | 75 | 10 | 15 | — | Stable |
| Sample 12 | 75 | 15 | 10 | — | Stable |
| Sample 13 | 65 | — | 17.5 | 17.5 | Stable |

Obtained polymer compositions of Samples 1-13 were then individually formulated in to a standard shampoo chassis according to Table-2.

TABLE 2
Standard shampoo formulation.

| | |
|---|---|
| Ingredients | % w/w |

Phase A

| | |
|---|---|
| Aqua (Deionized Water) | Add 100% |
| Mica and Titanium oxide | 0.10 |
| Suspending polymer composition (Hydroxypropyl | 0.50 |
| Methylcellulose (and) Cellulose Gum (and) Xanthan Gum) | |
| Sodium hydroxide (33%) | 0.03 |
| Cocamidopropyl Betaine (30%) | 6.67 |
| Sodium Laureth (2EO) Sulfate (28%) | 42.86 |

Phase B

| | |
|---|---|
| Aqua (Deionized Water) | 5.00 |
| Guar Hydroxypropyltrimonium Chloride | 0.20 |

Phase C

| | |
|---|---|
| Dimethiconol (and) TEA-Dodecylbenzenesulfonate | 3.00 |
| Phenoxyethanol and Caprylyl Glycol | 1.00 |
| Perfume | 0.50 |

Phase D

| | |
|---|---|
| Citric Acid | a.n. |
| Sodium Chloride | a.n.(1%) |
| Total | 100.00 |

*a.n: denotes ‘as needed’

Method for preparing shampoo composition of Table-2: Mica and titanium oxide were dispersed in water, suspending polymer composition according to Table-1 was added and mixed well until lump free. Sodium hydroxide was then added to the mixture to maintain a pH level of greater than 8.5 and mixed until fully hydrated, followed by cocamidopropyl betaine, sodium laureth (2EO) sulfate in the right order as given in Phase A. Guar hydroxypropyltrimonium chloride of Phase B was then dispersed in deionized water and added to Phase A. Ingredients of Phase C were then added in the right order while stirring at a constant speed of 500 rpm. The final pH of the composition was adjusted to a level of 5.5 to 6.5 by adding sodium hydroxide (33%) or citric acid (50%) and sodium chloride as given in Phase D to achieve a viscosity of 5000 to 8000 mPa·s. Shampoo compositions including suspending polymer compositions of Sample 1-13 were separately prepared using above method.

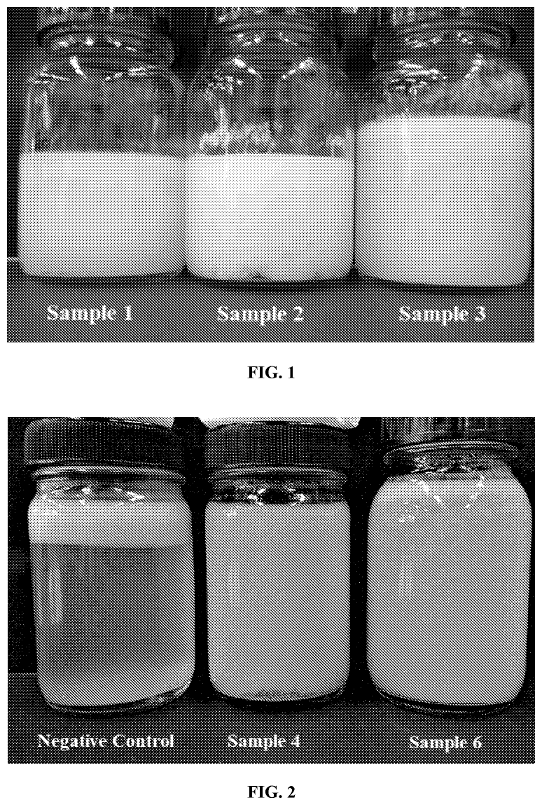

The stability of prepared shampoo compositions was tested using an accelerated stability test, performed according to the standard test procedure ISO/TR 13097:2013 in a temperature and humidity controlled oven at a temperature of 50° C. for a period of one month. Stability test results for shampoo compositions including suspending polymer compositions of Samples 1-3 are provided in FIG. 1. It was observed that all shampoo compositions were unstable for a period of less than 30 days at a temperature of 50° C.

Stability test results for shampoo compositions prepared using polymer compositions of Sample 4 and Sample 6 are provided in FIG. 2 and it is clear that all shampoo compositions are unstable for a period of less than 30 days at a temperature of 50° C. Negative control sample shown in FIG. 2 refers to a shampoo composition prepared without using any suspending polymer composition of present invention.

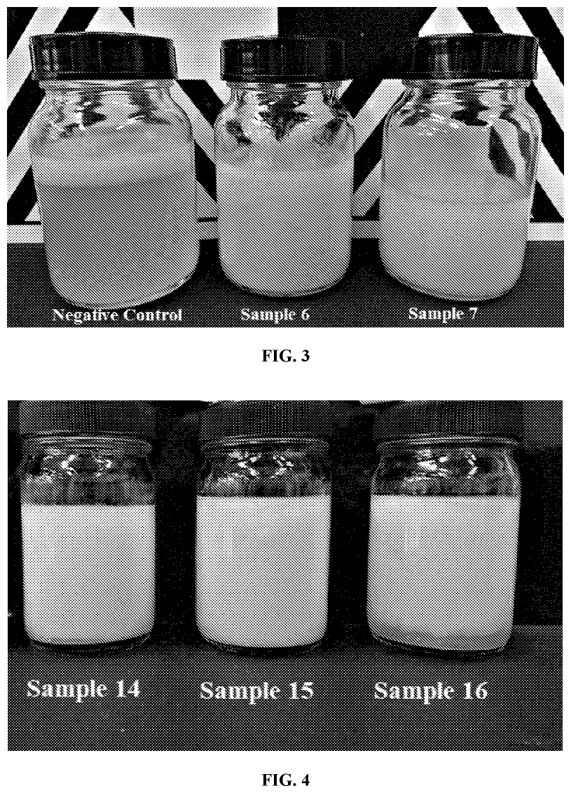

Stability test results for shampoo compositions prepared using polymer compositions of Samples 6-7 are provided in FIG. 3 and it is clear that the shampoo composition comprising suspending polymer composition of Sample 7 is highly stable for a period of 30 days at a temperature of 50° C. Negative control sample shown in FIG. 3 refers to a shampoo composition prepared without using any suspending polymer composition of this invention.

From the test results shown in FIG. 1-FIG. 3, it is evident that to achieve required suspension stability the suspending polymer composition consisting of a blend of all 3 polymers (hydroxypropylmethylcellulose (HPMC), xanthan gum, and carboxymethyl cellulose (CMC)) are essentially required at specific ratios.

### Example-2: Role of Degree of Substitution Values of Carboxymethyl Cellulose (CMC)

Hydroxypropylmethylcellulose (HPMC), xanthan gum, and carboxymethyl cellulose (CMC) were weighed in a bottle according to their respective w/w ratios as given in Table-3. The mixture of ingredients in powder form was then subjected to homogenization in IKA ROLLER 6 apparatus for 30 minutes at a speed of 80 rpm. Different grades of carboxymethyl cellulose with degree of substitution ranges according to Table-3 (less than 0.65, from 0.65 to 0.90, from 0.80 to 0.95 and from 1.12 to 1.21) were used in the preparation. Homogenized polymer compositions of Samples 14-16 were obtained using the same method.

TABLE 3
suspending polymer compositions with different degree of
substitution values of carboxymethyl cellulose (CMC).

| | | | CMC | CMC | CMC | CMC | |
|---|---|---|---|---|---|---|---|
| | | Xanthan | DS | 0.65- | 0.80- | 1.12- | |
| | HPMC | gum | <0.65 | 0.90 | 0.95 | 1.21 | |
| | (% | (% | (% | (% | (% | (% | |
| Samples | w/w) | w/w) | w/w) | w/w) | w/w) | w/w) | Stability |
| Sample 7 | 65 | 17.5 | 17.5 | — | — | — | Stable |
| Sample 14 | 65 | 17.5 | — | 17.5 | — | — | Unstable |
| Sample 15 | 65 | 17.5 | — | — | 17.5 | — | Unstable |
| Sample 16 | 65 | 17.5 | — | — | — | 17.5 | Unstable |

Polymer compositions of Samples 14-16 were then individually formulated into a standard shampoo chassis according to Table-2. The stability of prepared shampoo compositions was tested using an accelerated stability test, performed according to the standard test procedure ISO/TR 13097:2013 in a temperature and humidity controlled oven at a temperature of 50° C. for a period of one month.

The test results for shampoo compositions comprising suspending polymer compositions of Sample 14-16 are provided in FIG. 4 and it is clear that all the shampoo compositions are unstable in less than 30 days at 50° C. It was observed from the test results that the degree of substitution values less than 0.65 has proven best suspension stability compared to that of samples containing carboxy methyl cellulose grades having higher degree of substitutions. Further, a more degree of instability is observed with samples containing carboxymethyl cellulose grades of higher degree of substitutions.

### Example-3: Role of HPMC Vs MHEC or MC

Hydroxypropylmethylcellulose (HPMC), xanthan gum, and carboxymethyl cellulose (CMC) were weighed in a bottle according to their respective w/w ratios given in Table-4. The mixture of ingredients in powder form was then subjected to homogenization in IKA ROLLER 6 apparatus for 30 minutes at a speed of 80 rpm. Homogenized polymer compositions of Samples 17-18 were obtained using the same method.

TABLE 4
suspending polymer compositions—
Role of HPMC vs MHEC or MC.

| | | | | CMC | | |
|---|---|---|---|---|---|---|
| | | | | DS | Xanthan | |
| | HPMC | MHEC | MC | <0.6 | Gum | |
| | (% | (% | (% | (% | (% | |
| Samples | w/w) | w/w) | w/w) | w/w) | w/w) | Stability |
| Sample 7 | 65 | — | — | 17.50 | 17.50 | stable |
| Sample 17 | — | — | 65 | 17.50 | 17.50 | unstable |
| Sample 18 | — | 65 | — | 17.50 | 17.50 | unstable |

The polymer compositions of Samples 17-18 were then individually formulated in to a standard shampoo chassis according to Table-2. The stability of prepared shampoo compositions was tested using an accelerated stability test, performed according to the standard test procedure ISO/TR 13097:2013 in a temperature and humidity controlled oven at a temperature of 50° C. for a period of one month.

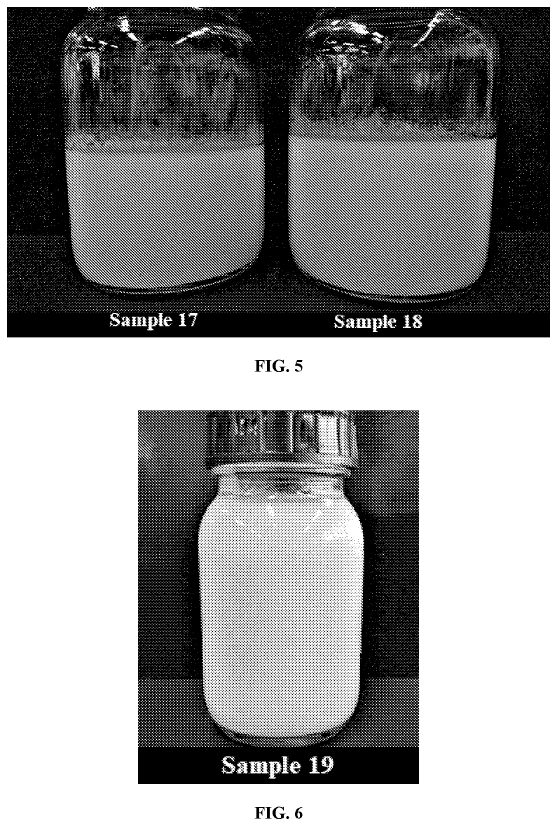

It was observed from the test results that the suspending polymer compositions including HPMC has proven best possible suspending performance over compositions of other polymer based samples (MHEC or MC), when used in combination with Xanthan gum and carboxymethyl cellulose having degree of substitution of less than 0.65. The test results for shampoo compositions comprising Samples 17-18 are provided in FIG. 5 and it is clear that all the shampoo compositions are unstable in a period of less than 30 days at a temperature of 50° C.

### Example-4: Shampoo Composition

A suspending polymer composition of Sample 7 was formulated into a shampoo composition according to Table-5.

TABLE 5
Shampoo formulation

| | |
|---|---|
| Ingredients | % w/w |

Phase A

| | |
|---|---|
| Aqua (Deionized Water) | Add 100% |
| Mica and Titanium oxide | 0.10 |
| Suspending polymer composition | 0.50 |
| of Sample 7 (Hydroxypropyl Methylcellulose | |
| (and) Cellulose Gum (and) Xanthan Gum) | |
| Sodium hydroxide (33%) | 0.03 |
| Cocamidopropyl Betaine (30%) | 6.67 |
| Sodium Laureth (2EO) Sulfate (28%) | 42.86 |

Phase B

| | |
|---|---|
| Aqua (Deionized Water) | 5.00 |
| Guar Hydroxypropyltrimonium Chloride | 0.20 |

Phase C

| | |
|---|---|
| Dimethiconol (and) TEA-Dodecylbenzenesulfonate | 3.00 |
| Phenoxyethanol and Caprylyl Glycol | 1.00 |
| Perfume | 0.50 |

Phase D

| | |
|---|---|
| Citric Acid | a.n. |
| Sodium Chloride | a.n.(1%) |
| Total | 100.00 |

*a.n: denotes ‘as needed’

Method for preparing shampoo composition of Table-5: Mica and titanium oxide were dispersed in water, suspending polymer composition of Sample 7 was added and mixed well until lump free. Sodium hydroxide was then added to the mixture to maintain a pH level of greater than 8.5 and mixed until fully hydrated, followed by cocamidopropyl betaine, sodium laureth (2EO) sulfate in the right order as given in Phase A. Guar hydroxypropyltrimonium chloride of Phase B was then dispersed in deionized water and added to Phase A. Ingredients of Phase C were then added in the right order while stirring at a constant speed of 500 rpm. The final pH of the composition was adjusted to a level of 5.5 to 6.5 by adding sodium hydroxide (33%) or citric acid (50%) and sodium chloride as given in Phase D to achieve a viscosity of 5000 to 8000 mPa·s.

The stability of prepared shampoo compositions was tested using an accelerated stability test, performed according to the standard test procedure ISO/TR 13097:2013 in a temperature and humidity controlled oven at a temperature of 50° C. for a period of one month and found stable. The prepared shampoo composition was also found stable in a stability test, performed according to the standard test procedure ISO/TR 18811:2018 in a temperature and humidity controlled oven at a temperature of 45° C. for a period of three months (industry benchmark test method in temperature and humidity controlled ovens).

### Example-5: Anti-Dandruff Shampoo Formulation

A suspending polymer composition of Sample 7 was formulated into an anti-dandruff shampoo composition according to Table-6 (Sample 19)

TABLE 6
Anti-dandruff shampoo formulation

| | |
|---|---|
| Ingredients | % w/w |

Phase A

| | |
|---|---|
| Aqua (Deionized Water) | ad 100% |
| Suspending polymer composition | 0.60 |
| of Sample 7 (Hydroxypropyl Methylcellulose | |
| (and) Cellulose Gum (and) Xanthan Gum) | |
| Sodium hydroxide (33%) | 0.03 |
| Cocamidopropyl Betaine (30%) | 6.67 |
| Sodium Laureth(2EO) Sulfate (28%) | 42.86 |

Phase B

| | |
|---|---|
| Aqua (Deionized Water) | 5.00 |
| Guar Hydroxypropyltrimonium Chloride | 0.20 |

Phase C

| | |
|---|---|
| Phenoxyethanol and Caprylyl Glycol | 1.00 |
| Perfume | 0.50 |
| Dimethiconol (and) TEA-Dodecylbenzenesulfonate | 3.00 |
| Zinc Pyrithione (48%) | 2.00 |

Phase D

| | |
|---|---|
| Citric Acid | a.n. |
| Sodium Chloride | a.n.(1%) |
| Total | 100 |

*a.n-denotes needed

Method for preparing anti-dandruff shampoo formulation of Table-6: Suspending polymer composition of Sample 7 (rheology modifier) was added in to water and mixed until lump free. Sodium hydroxide was then added to the mixture until reaching a pH level of greater than 8.5 and mixed well at 500 rpm until fully hydrated. Cocamidopropyl betaine, sodium laureth (2EO) sulfate and other ingredients of Phase A were added in the right order. Guar hydroxypropyltrimonium chloride of Phase B was then dissolved in deionized water until lump free and added to Phase A. Rest of the ingredients were then added in right order as given in Phase C. The final pH of the composition was adjusted to a level of 5.5 to 6.5 by adding sodium hydroxide (33%) or citric acid (50%) and sodium chloride as given in Phase D to achieve a viscosity of 5000 to 8000 mPa·s. An anti-dandruff shampoo composition of Sample 19 was obtained according to above method.

The anti-dandruff shampoo composition of Sample 19 was then subjected to accelerated stability test, performed according to the standard test procedure ISO/TR 13097:2013 in a temperature and humidity controlled oven at a temperature of 50° C. for a period of one month. Test results are provided in FIG. 6 and it is clear that the anti-dandruff shampoo composition is highly stable after a period of one month at a temperature of 50° C.

### Example-6: Scentsual Fabric Conditioner Formulation

A suspending polymer composition of Sample 7 was formulated in to a scentsual fabric conditioner composition according to Table-7.

TABLE 7
Scentsual fabric conditioner formulation

| | | |
|---|---|---|
| | Positive Control | 0.20% Sample 7 |
| Ingredients | (% w/w) | (% w/w) |

Phase A

| | | |
|---|---|---|
| Deionized water | 90.82 | 91.12 |
| Poly (2-dimethylamino)ethyl methacrylate methyl chloride | 0.5 | 0.00 |
| quaternary salt | | |
| Dialkyl ammonium methosulfate | 5.55 | 5.55 |
| Suspending polymer composition of Sample 7 (Hydroxypropyl | 0 | 0.20 |
| Methylcellulose (and) Cellulose Gum (and) Xanthan Gum) | | |
| Blue Colorant (0.1% Active Solution) | 0.36 | 0.36 |

Phase B

| | | |
|---|---|---|
| C6-12 Alcohols Ethoxylated Propoxylated | | 0.50 |
| Seed Oil Alcohol Ethoxylate 9EO | 0.5 | |
| Fragrance oil | 0.5 | 0.50 |

Phase C

| | | |
|---|---|---|
| Microencapsulated fragrance (43.3% Active Solution) | 1.77 | 1.77 |
| Total | 100 | 100 |

Method for preparing scentsual fabric conditioner of Table-7: Poly (2-dimethylamino)ethyl methacrylate methyl chloride quaternary salt or the Suspending polymer composition of Sample 7 (Hydroxypropyl Methylcellulose (and) Cellulose Gum (and) Xanthan Gum) of Phase A was added in to deionized water and mixed until lump free, it was then heated to a temperature of 60° C., dialkyl ammonium methosulfate was then added and stirred until dispersed well. Perfume of Phase B is then dissolved in C6-12 alcohols ethoxylated propoxylated (and) seed oil alcohol ethoxylate 9EO and added to phase A. Captivates (Microencapsulated fragrance) of Phase C were then added with slow stirring. Scentsual fabric conditioner compositions of Sample 20 and a positive control sample were obtained using above method. A negative control sample was also prepared by the same formulation and method, excluding the ingredient rheology modifier of Phase A.

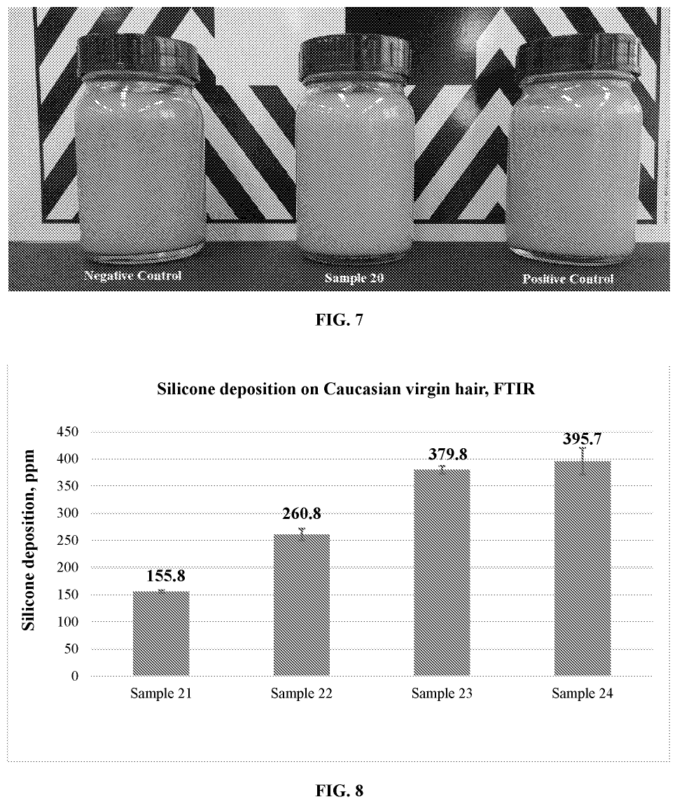

The scentsual fabric conditioner compositions of Sample 20, negative control sample, and positive control sample were then subjected to an accelerated stability, performed according to the standard test procedure ISO/TR 13097:2013 in a temperature and humidity controlled oven at a temperature of 50° C. for a period of one month. Test results are provided in FIG. 7 and it is clear that the fabric conditioner composition is highly stable after a period of one month at a temperature of 50° C.

### Example-7: Conditioning Performance and Silicone Deposition

Acrylate-alkali soluble emulsion chemistries such as Carbomer are widely used in shampoo compositions to suspend conditioning agents such as silicones. One of the reasons for their popularity includes, these kinds of formulations do not offer any negative influence on the silicone deposition on hair. Hydrophobically modified alkali-soluble emulsion (HASE) chemistries such as acrylate copolymers are also used in shampoos on rinse-off systems due to their superior thickening and suspension properties. They are well known though to prevent the silicone deposition out of the conditioning shampoo to hair.

In this example, conditioning shampoo compositions of Samples 21-26 were prepared using suspending polymers according to Table-8 and as per the standard shampoo formulation provided in Table-2. This formulation also uses a larger particle size silicone (0.5 micron) as water insoluble ingredient.

TABLE 8
Conditioning shampoo compositions

| Samples | Composition |
|---|---|
| Sample 21 | Conditioning shampoo with 1.50 wt. % Acrylates Copolymer |
| Sample 22 | Conditioning shampoo with 0.50 wt. % Xanthan Gum |
| Sample 23 | Conditioning shampoo with 0.50 wt. % Sample 7 |
| Sample 24 | Conditioning shampoo with 0.40 wt. % Carbomer 980 |
| Sample 25 | Conditioning shampoo with 0.5 wt. % Carbomer 980 |
| Sample 26 | Conditioning shampoo with 0.4 wt. % Sample 8 |

The shampoo compositions of Samples 21-24 were then tested for quantified silicone deposition, measured on damaged hair with Attenuated Total Reflection-Fourier Transform Infrared Spectroscopy (ATR-FTIR) trace 100 from Shimadzu. Same test was repeated with shampoo compositions of Sample 23, and Samples 25-26 after subjected to one shampoo treatment. It is clear from the test results that the silicone deposition of shampoo compositions including current suspending polymer composition of Sample 7 is comparable to the carbomer based systems and superior when compared to the acrylates chemistries. It can also be seen that both acrylates copolymer and pure xanthan gum-based samples exhibit a negative influence on the silicone deposition as evident from FIG. 8 and FIG. 9.

### Example-8: Dry Friction

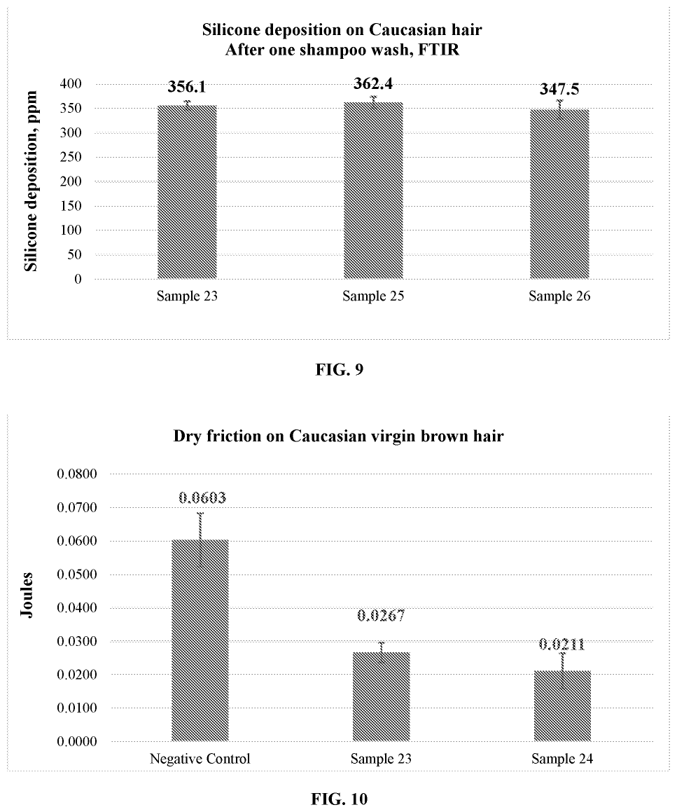

The silicone deposition can be correlated with the hair surface smoothness, which can be instrumentally measured by measuring the dry friction. In this example, the dry friction of Samples 23-24 of Example-7 on Caucasian virgin brown hair were measured using a Diastron MTT 175 apparatus. Test results are provided in FIG. 10, and it is evident from the chart that the dry friction of shampoo compositions including current suspending polymer composition of Sample 7 is superior to the carbomer based systems. A Negative control sample indicates the same test performed on a non-conditioning shampoo (12:2 Sodium laureth sulfate:cocamidopropyl betaine (SLES:CAPB), without using any conditioning polymers).

### Example-9: Preferred Foam Vs Xanthan Gum

One of the important parameters in the rinse-off systems is the foam. Preferable rheology modifiers do not offer any influence on foam properties. In this example, the foam height of shampoo samples prepared by 10% solutions of shampoo compositions of Samples 22-24 in water were measured using a Kruss DFA 100 foam analyzer.

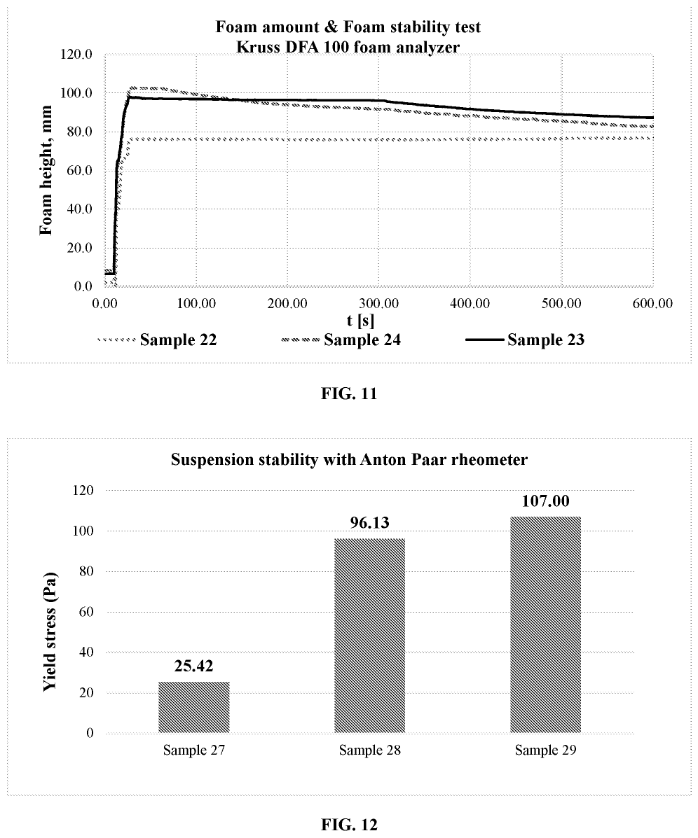

Test results are provided in FIG. 11. It is evident that the xanthan gum has an influence on the foam type and foam stability, that can be related to a more difficult to rinse-off type shampoo compositions. It can also be observed from the chart that xanthan gum has a negative influence on the initial foam reducing the number of bubbles. On the other hand, it makes over time the foam very stable. Highly stable foam indicates a difficult to rinse-off performance. It can be clearly seen from the chart that shampoo compositions including the suspending polymer composition of Sample 7 has virtually no influence on the foam properties.

### Example-10: Sulfate-Free Shampoo Formulation

The suspending polymer composition of Sample 7 was formulated into a sulfate-free shampoo formulation according to Table-9.

TABLE 9
Sulfate-free shampoo formulation.

| | |
|---|---|
| Ingredients | % w/w |

Phase A

| | |
|---|---|
| Aqua (Deionized Water) | ad 100% |
| Suspending polymer composition | 0.50 |
| of Sample 7 (Hydroxypropyl Methylcellulose | |
| (and) Cellulose Gum (and) Xanthan Gum) | 0.5 |
| Guar Hydroxypropyltrimonium Chloride | |
| Sodium hydroxide (33%) | 0.03 |

Phase B

| | |
|---|---|
| Sodium Cocoyl Isethionate | 8.00 |
| Cetyl Alcohol | 1.0 |

Phase C

| | |
|---|---|
| Cocamidopropyl Betaine (30%) | 6.67 |
| Decyl Glucoside | 6.50 |
| Sodium Lauroyl Methyl Isethionate | 7.50 |
| Octyldodecanol (and) Santalum album (Sandalwood) wood | 1.00 |
| extract | |

Phase D

| | |
|---|---|
| Benzyl alcohol (and) aqua (water) (and) | 1.00 |
| sodium Benzoate (and) Potassium sorbate | |
| Parfum | 0.30 |

Phase E

| | |
|---|---|
| Citric acid (50%) | 0.50 |
| Total | 100 |

Following samples were prepared from the sulfate-free shampoo formulation of Table-9 and subjected to suspension stability test using Anton Paar rheometer.

| Samples | Composition |
|---|---|
| Sample 27 | Sulfate-free negative shampoo without rheology modifier |
| Sample 28 | Sulfate-free shampoo with 0.50 wt. % Carbomer |
| Sample 29 | Sulfate-free shampoo with 0.5 wt. % Sample 7 |

### Example-11: Yield Stress Measurement Using Anton Paar Rheometer-Evaluation of Suspension Stability

The sulfate-free shampoo compositions of sample 27-29 were studied with Anton Paar Rheometer to evaluate the suspension stability using yield stress measurement. For this test, the shear strain is preset from 0.01-1000% at a constant angular frequency of 10%.

Test results are provided in FIG. 12-14. It can be seen from FIG. 12 that the sulfate-free shampoo composition of sample 29 exhibits significantly high yield stress value compared to other samples. This clearly indicates the improvement in suspension stability of sample 29. Test results also shows that sample 29 performs better than carbomer containing sulfate-free shampoo composition of sample 28.

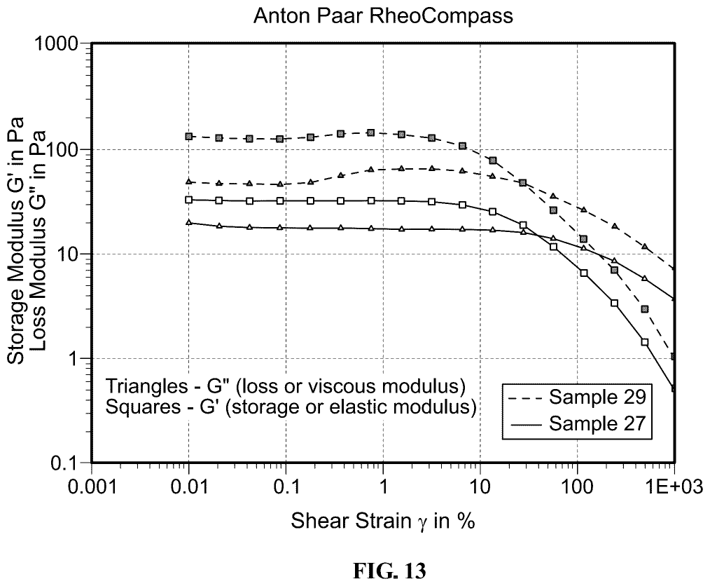

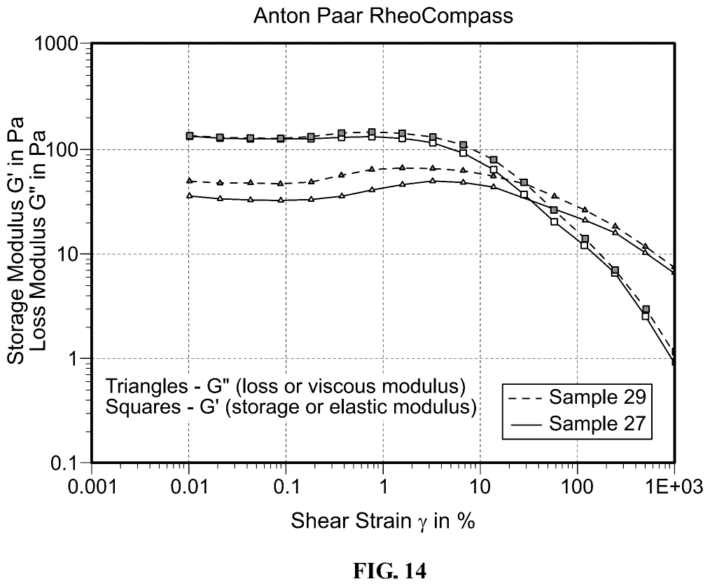

FIG. 13-14 shows the performance of sample 27-29 across measuring shear range using Anton Paar Rheometer. Triangular shaped data points indicate loss modulus or viscous modulus (G″) and square shaped data points indicate storage modulus or elastic modulus (G′). The graphs show a clear domination of elastic behavior of samples over the viscous behavior (G′>G″) and exhibits viscoelastic gel-like structure. Increase in the yield stress also indicates a significant improvement in the structural strength. Performance of sample 29 across measuring shear range exhibits superior suspension stability over sample 27-28.

### Example-12: Temperature Stability Test

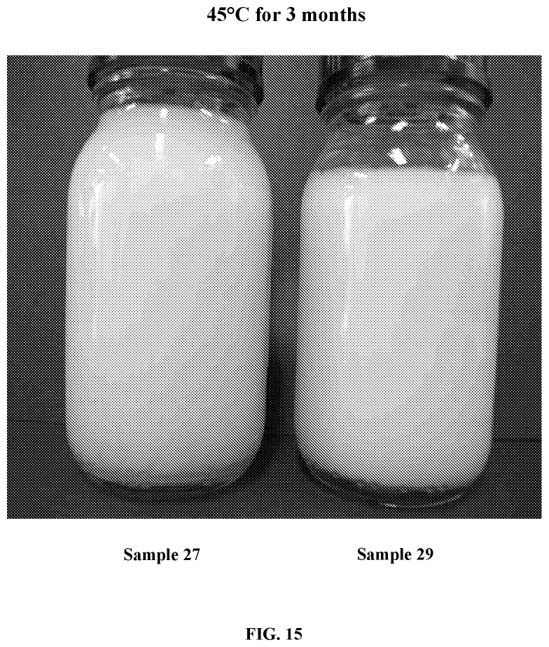

The sulfate-free shampoo compositions of sample 27-29 were then subjected to a conventional stability test at (i) 5° C., (ii) Room Temperature, and (iii) 45° C. for 3 months, performed according to the standard test procedures ISO/TR 13097:2013 and ISO/TR 18811:2018 in a temperature and humidity controlled oven and the results are provided in FIG. 15. It can be clearly seen from the image that the sample 27 has a breaking in texture with water layer at the bottom when subject to a stability test at 45° C. for 3 months and, sample 29 did pass all 3 levels of the temperature stability tests and maintains a homogeneous texture without any evidence of phase separation.

### Example-13: Large Amplitude Oscillation Shear (LAOS) Using Anton Paar Rheometer—Evaluation of Product Texture

The sulfate-free shampoo compositions of samples 27-29 were studied with Anton Paar rheometer to evaluate the product texture with large amplitude oscillation shear measurement (LAOS measurement). When exceeding the LVE range, both the viscous and elastic behavior of the sample material will change at large amplitudes and the deformation of microstructure can be large and rapid. Therefore, the nonlinear structural properties will control system response. Consequently, large amplitude oscillatory shear tests are required to investigate the nonlinear viscoelastic behavior of a complex fluid. For this test, an appropriate range of strain amplitude from 10-800% is set at a constant frequency of 50 (rad/s) to investigate the change in microstructure of a complex fluid in nonlinear viscoelastic region.

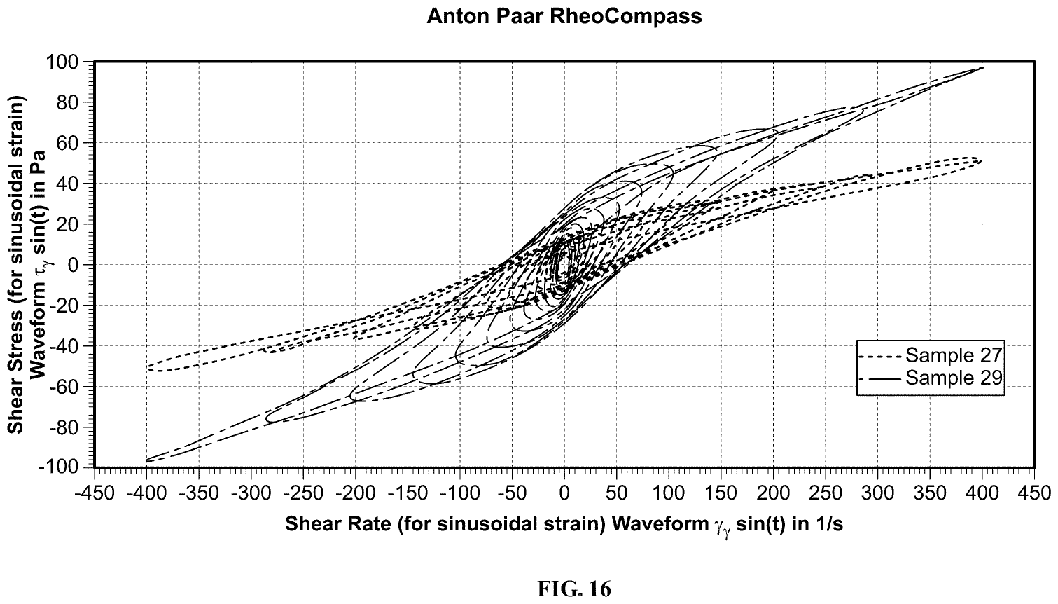

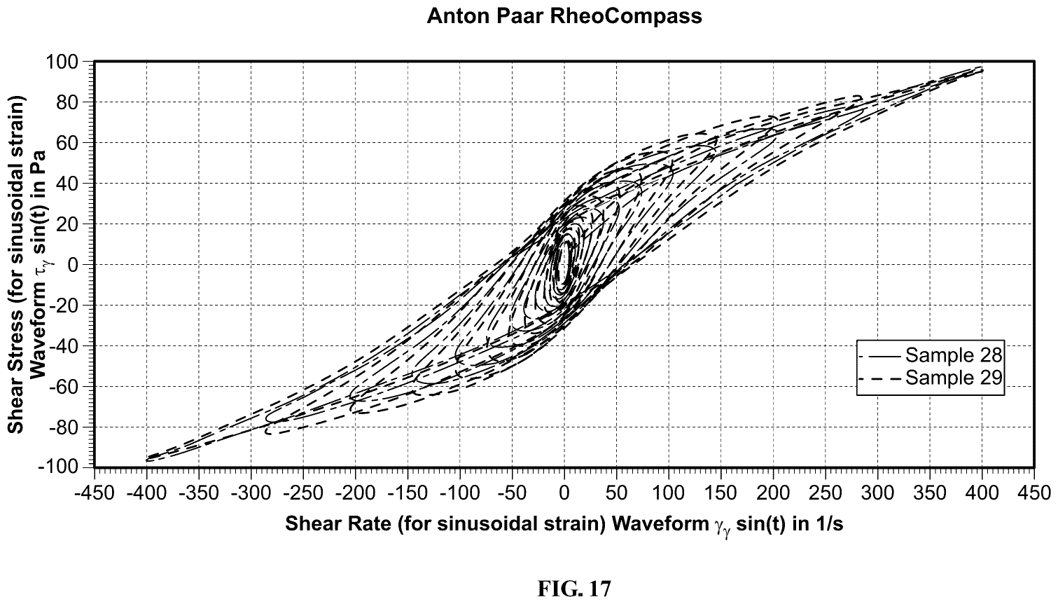

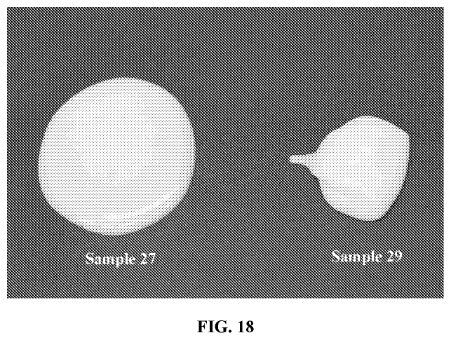

According to FIG. 16, sample 29 shows a high viscoelastic behavior at low and middle shear (<200 1/s), viscous behavior at high shear and a high overall shear stress; sample 27 shows low viscoelastic behavior at low and middle shear (<200 1/s), a high viscous behavior at high shear and a low overall shear stress. FIG. 17 also show similar performance of sample 29 across shear range, similar overall stress, and a high viscoelastic behavior at low and middle shear. The sample also turned into fluid-like behavior at high shear.

The Lissajous curves from test results provide qualitative insights on the rheological behavior. It is clear from Lissajous diagram that the addition of sample 29 in sulfate-free shampoo base has significantly increased the viscoelastic behavior (in the form of ellipses) of test compositions. This indicates a significant contribution in the creaminess for shampoo texture. Further, the increase in consistency of shampoo texture with sample 29 was exhibiting with significantly high overall stress (100 Pa) compared to sample 27 (50 Pa).

It is also evident from the test results that the product texture of shampoo containing sample 29 was comparable or even slightly better than shampoo containing acrylates-based (carbomer) polymer of sample 28. It was indicated with the similar performance in overall shear stress and across measuring shear range. At high shear, shampoo composition with sample 29 performed likely as sample 28, showing the ease of turning into fluid-like. This indicated in the form of narrow loops at high shear (>250 1/s) and are evident for the ease of rinsability of test shampoos.

### Example-14: Evaluation of Texture of the Sulfate-Free Shampoo Compositions

The sulfate-free shampoo compositions of sample 27 and sample 29 were studied by visual examination. The textures of samples were evaluated by observing the spreading behavior of shampoo samples poured on to a plain surface. FIG. 18 shows a significant difference in the texture of sample 29 and the watery texture cause to develop a consistent and creamy gel.

### Example-15: Flow Profile Measurement Using Anton Paar Rheometer-Evaluation of Thickening Effect

The sulfate-free shampoo compositions of sample 27-29 were studied with Anton Paar Rheometer to evaluate the flow behavior using rheology test with a controlled range of shear rate. For this test, the shear rate is preset from 0.1 (1/s)-10000 (1/s) and the viscosity profile or flow profile is measured as a function of shear rate.

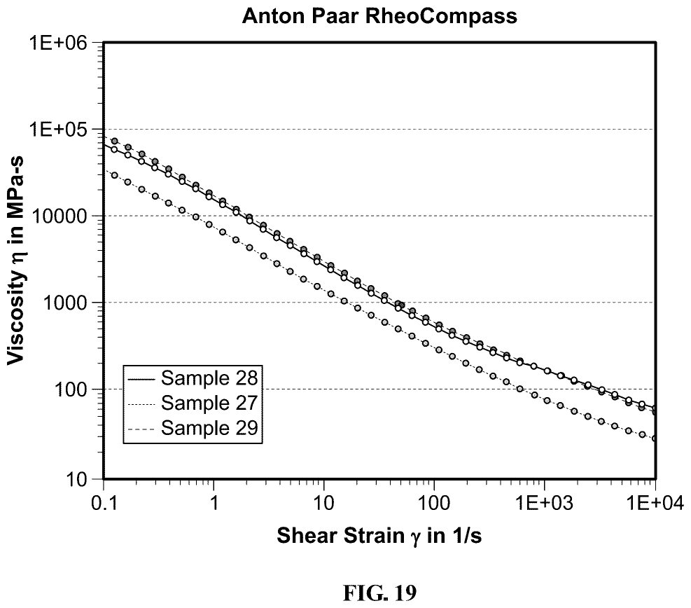

Test results are provided in FIG. 19, and a shear-thinning behavior is evident from the graph for all the test compositions of samples 27-29. This flow property is desired for all hair shampoo formulations. Sample 29 clearly showed the thickening effect compared to sample 27 indicated with significant increase in the initial viscosity and performed as good as acrylates-based (carbomer) polymer or even slightly better.

### Example-16: Synergistic Effect on Viscosity of Shampoo Composition with Addition of Sodium Chloride

In this example, the effect of viscosity of following samples, prepared from the shampoo formulation of Table-5 (Example-4), were measured while adding sodium chloride at various dosage levels mentioned in the table below. Sodium chloride is a common viscosifying agent in a 12:2 Sodium laureth sulfate:cocamidopropyl betaine (SLES:CAPB) based surfactant shampoo systems.

| Samples | Composition |
|---|---|
| Sample 30 | Shampoo composition with 0.5 wt. % Sample 7 |
| Sample 31 | Negative shampoo composition without rheology modifier |

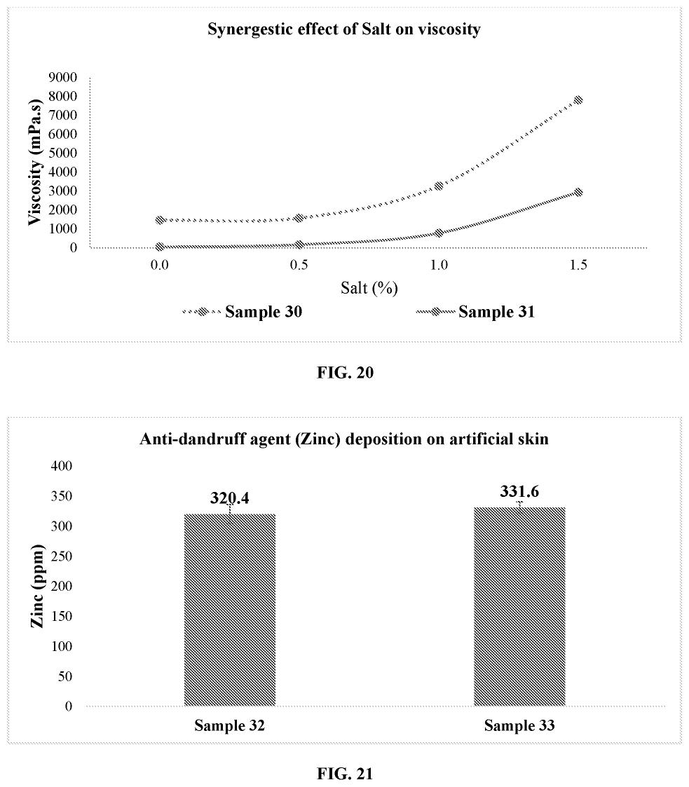

Test results are provided in the table below and also in FIG. 20, and it is evident from the chart that there is a synergistic effect on the viscosity of the shampoo composition in combination with various dosages of sodium chloride.

| | |
|---|---|
| Salt added to | Viscosity Measurements |

| shampoo (wt. %) | Sample 30 | Sample 31 |
|---|---|---|
| 0.0 | 1460 | 50 |
| 0.5 | 1570 | 170 |
| 1.0 | 3259 | 779 |
| 1.5 | 7798 | 2929 |

### Example-17: Evaluation of Anti-Dandruff Agent (Zinc) Deposition Behavior

Following samples prepared using the anti-dandruff shampoo formulation of Table-6 (Example-5), having similar dose levels (0.6 wt. %) of suspending polymer composition of Sample 7 and Carbomer.

| Samples | Composition |
|---|---|
| Sample 32 | Anti-dandruff shampoo composition with 0.6 wt. % Sample 7 |
| Sample 33 | Anti-dandruff shampoo composition with 0.6 wt. % Carbomer |

The Zinc deposition performance of anti-dandruff shampoo compositions of samples 32-33 were measured by quantifying the amount of Zinc on Vitro-Skin by XRF (X-ray fluorescence spectrometry) analysis. To control the results, 4 reference standards of Zinc Sulfate heptahydrate were included in each study. Standard XRF-cups with artificial skin were treated with diluted shampoo samples, rinsed, and the dried for XRF measurement. The Zinc deposition values were then calculated to ppm of Zinc.

Test results are provided in FIG. 21, and it is evident that the anti-dandruff agent deposition behavior of shampoo composition of sample 32 having 0.6 wt. % suspending polymer composition of Sample 7 is comparable with that of shampoo composition of sample 33 having 0.6 wt. % Carbomer and there is no proof of any negative effect of sample 32 on the deposition of the anti-dandruff agent on the scalp.

What is claimed is:

1. A suspending polymer composition comprising a blend of:
(i) about 50 wt. % to about 75 wt. % of hydroxypropylmethylcellulose (HPMC);
(ii) about 10 wt. % to about 25 wt. % of xanthan gum; and,
(iii) about 10 wt. % to about 25 wt. % of carboxymethyl cellulose (CMC).

2. The composition according to claim 1,
wherein the amount of hydroxypropylmethylcellulose (HPMC) is about 65 wt. %,
wherein the amount of xanthan gum is about 17.5 wt. %, and,
wherein, the amount of carboxymethyl cellulose (CMC) is about 17.5 wt. %.

3. The composition according to claim 1, wherein the carboxymethyl cellulose (CMC) has a degree of substitution (DS) of less than 0.65 or less than 0.60.

4. The composition according to claim 1 wherein the composition is used in personal care compositions and home care compositions.

5. A personal care composition comprises:
(a) about 0.05 wt. % to about 5 wt. % of a suspending polymer composition comprising a blend of: (i) about 50 wt. % to about 75 wt. % of hydroxypropylmethylcellulose (HPMC); (ii) about 10 wt. % to about 25 wt. % of xanthan gum; and (iii) about 10 wt. % to about 25 wt. % of carboxymethyl cellulose (CMC);
(b) about 0.1 wt. % to about 30 wt. % of at least one surfactant selected from the group consisting of anionic, cationic, nonionic, zwitterionic, amphoteric and combinations thereof;
(c) about 0.1 wt. % to about 10 wt. % of at least one personal care ingredient; and,
(d) about 30 wt. % to about 70 wt. % water based on the total weight of the composition,
wherein the composition is free from acrylic acids, methacrylic acids, or derivatives thereof.

6. The composition according to claim 5,
wherein the amount of hydroxypropylmethylcellulose (HPMC) is about 65 wt. %;
wherein the amount of xanthan gum is about 17.5 wt. %;
wherein the amount of carboxymethyl cellulose (CMC) is about 17.5 wt. %.; and,
wherein the carboxymethyl cellulose (CMC) has a degree of substitution (DS) of less than 0.65 or less than 0.60.

7. The composition according to claim 5, wherein the personal care ingredient is selected from the group consisting of water-insoluble ingredients, oxidizing agents, conditioning agents, humectants, pH adjusting buffers, waxes, mineral oils, emulsifiers, fragrances, preservatives, fatty substances, gelling agents, thickeners, moisturizers, emollients, hydrophilic or lipophilic active agent, antioxidants, sequestering agents, acidifying or basifying agents, fillers, dyestuffs, plant extracts, proteins, peptides, neutralizing agents, solvents, anti-dandruff ingredients, reducing agents and combinations thereof.

8. The composition according to claim 7, wherein the water-insoluble ingredient is selected from the group consisting of silicones, natural and synthetic oils, emollients, organic and/or inorganic pearlescent pigments, beads, fatty alcohols, discrete particles, polymers, and combinations thereof.

9. The composition according to claim 5, wherein the personal care composition is formulated into a spray, a lotion, a mousse, a fluid, a serum, a solution, a perm, an emulsion, a gel, a vesicle a dispersion, a paste, a cream, a solid stick, a shampoo, a balm, a wipe, a milk, a foam or a jelly.

10. The composition according to claim 5, wherein the personal care composition is selected from the group consisting of hair-care products, shampoos, hair conditioners, leave in and rinse off conditioners, styling and treating hair compositions, hair perming products, hair relaxants, hair straighteners, hair sprays and lacquers, permanent hair dyeing systems, hair styling mousses, hair gels, semi-permanent hair dyeing systems, temporary hair dyeing systems, hair bleaching systems, permanent hair wave systems, hair setting formulations, skin-care products, bath products, shower products, liquid soaps, bar soaps, fragrances and/or odoriferous ingredients consisting preparations, deodorizing and antiperspirant preparations, decorative preparations, light protection formulations, shaving lotions, body oils, body lotions, body gels, treatment creams, body cleaning products, skin protection ointments, shaving and aftershave preparations, skin powders, lipsticks, nail varnishes, eye shadows, mascaras, dry and moist make-up, rouge, powders, depilatory agents and sun care products.

11. A shampoo composition comprising:
(a) about 0.05 wt. % to about 5 wt. % of suspending polymer composition comprising a blend of (i) about 50 wt. % to about 75 wt. % of hydroxypropylmethylcellulose (HPMC); (ii) about 10 wt. % to about 25 wt. % of xanthan gum; (iii) about 10 wt. % to about 25 wt. % of carboxymethyl cellulose (CMC);
(b) about 0.1 wt. % to about 30 wt. % of at least one surfactant selected from the group consisting of anionic, cationic, nonionic, zwitterionic, amphoteric and combinations thereof;
(c) about 0.1 wt. % to about 10 wt. % of at least one shampoo ingredient; and,
(d) about 30 wt. % to about 70 wt. % water based on the total weight of the composition.
wherein the composition is free from acrylic acids, methacrylic acids, or derivatives thereof.

12. The composition according to claim 11, wherein the surfactant comprises an anionic surfactant selected from the group consisting of alkyl sulfates and alkyl ether sulfates or a zwitterionic surfactant selected from the group consisting of betaines, and combinations thereof.

13. The composition according to claim 11, wherein the composition is a 2-in-1 shampoo composition, a 3-in-1 shampoo composition, a sulfate free shampoo composition, a silicone-free shampoo composition, an antidandruff shampoo composition, or a natural shampoo composition.

14. The composition according to claim 11, wherein the shampoo ingredient is selected from the group consisting of water-insoluble ingredients, oxidizing agents, conditioning agents, humectants, pH adjusting buffers, waxes, mineral oils, emulsifiers, fragrances, preservatives, fatty substances, gelling agents, thickeners, moisturizers, emollients, hydrophilic or lipophilic active agent, antioxidants, sequestering agents, acidifying or basifying agents, fillers, dyestuffs, plant extracts, proteins, peptides, neutralizing agents, solvents, anti-dandruff ingredients, reducing agents and combinations thereof.

15. A home care composition comprising:
(a) about 0.05 wt. % to about 5 wt. % of a suspending polymer composition comprising a blend of: (i) about 50 wt. % to about 75 wt. % of hydroxypropylmethylcellulose (HPMC); (ii) about 10 wt. % to about 25 wt. % of xanthan gum; and (iii) about 10 wt. % to about 25 wt. % of carboxymethyl cellulose (CMC);
(b) about 0.1 wt. % to about 30 wt. % of at least one surfactant selected from the group consisting of anionic, cationic, nonionic, zwitterionic, amphoteric and combinations thereof;
(c) about 0.1 wt. % to about 10 wt. % of at least one home care ingredient; and,
(d) about 30 wt. % to about 70 wt. % water based on the total weight of the composition.

16. The composition according to claim 15,
wherein the amount of hydroxypropylmethylcellulose (HPMC) is about 65 wt. %;
wherein the amount of xanthan gum is about 17.5 wt. %;
wherein the amount of carboxymethyl cellulose (CMC) is about 17.5 wt. %.; and,
wherein the carboxymethyl cellulose (CMC) has a degree of substitution (DS) of less than 0.65 or less than 0.60.

17. The composition according to claim 15, wherein the home care ingredient is selected from the group consisting of water-insoluble ingredients, rheology modifiers, solvents, phosphates, phosphonates, chelating agents, defoamers, anti-redeposition agents, fabric conditioners, fabric softeners, polish additives, plasticizers, colorants, preservatives, fragrances, microencapsulated fragrances, antifoam compounds, antibacterial or antifungal agents, abrasives, enzymes, optical brighteners, antioxidants, bleaching agents, emulsifiers, functional polymers, viscosifying agents, alcohols, oils, dye transfer inhibitors, hydrotropes, dye sequestrants, color fixatives, pH controlling agents, electrolytes, and combinations thereof.

18. The composition according to claim 15, wherein the home care composition is selected from the group consisting of laundry washing products, dish washing products, fabric conditioner, heavy duty cleaning products, disinfecting products, fabric enhancing products, fabric softener, fabric abrasion reducing products, toilet cleaning products, floor cleaning products, window cleaning products, auto polishing products, automobile cleaning products, shoe polishing products, household fragrance products, wall coloring products and wall paper adhesives.

19. A fabric conditioner composition comprising:
(a) about 0.05 wt. % to about 5 wt. % of suspending polymer composition comprising a blend of (i) about 50 wt. % to about 75 wt. % of hydroxypropylmethylcellulose (HPMC); (ii) about 10 wt. % to about 25 wt. % of xanthan gum; (iii) about 10 wt. % to about 25 wt. % of carboxymethyl cellulose (CMC);
(b) about 0.1 wt. % to about 30 wt. % of at least one surfactant selected from the group consisting of anionic, cationic, nonionic, zwitterionic, amphoteric and combinations thereof;
(c) about 0.1 wt. % to about 10 wt. % of at least one fabric care ingredient; and,
(d) about 30 wt. % to about 70 wt. % water based on the total weight of the composition.

20. The composition according to claim 19, wherein the surfactant is a nonionic surfactant selected from the group consisting of C6-12 alcohol alkoxylates, seed oil alcohol alkoxylates, and combinations thereof

21. The composition according to claim 19, wherein the fabric care ingredient is selected from the group consisting of water-insoluble ingredients, rheology modifiers, solvents, phosphates, phosphonates, chelating agents, defoamers, anti-redeposition agents, fabric conditioners, fabric softeners, polish additives, plasticizers, colorants, preservatives, fragrances, microencapsulated fragrances, antifoam compounds, antibacterial or antifungal agents, abrasives, enzymes, optical brighteners, antioxidants, bleaching agents, emulsifiers, functional polymers, viscosifying agents, alcohols, oils, dye transfer inhibitors, hydrotropes, dye sequestrants, color fixatives, pH controlling agents, electrolytes, and combinations thereof.
